(19) United States
(12) Patent Application Publication
(10) Pub. No.: US 2004/0082613 A1
(43) Pub. Date: Apr. 29, 2004

(54) Modulators of Cdk9 as a therapeutic target in cardiac hypertrophy

(76) Inventors: Michael D. Schneider, Houston, TX (US); Motoaki Sano, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/609,073

(22) Filed: Jun. 27, 2003

Related U.S. Application Data
(60) Provisional application No. 60/392,744, filed on Jun. 28, 2002.
(60) Provisional application No. 60/426,883, filed on Nov. 15, 2002.

(51) Int. Cl. A61K 31/452; A61K 31/401

(52) U.S. Cl. 514/320; 514/423

(57) ABSTRACT

The present invention relates generally to the field of cardiology. More particularly, the present invention relates to methods of using inhibitors of cyclin dependent kinase 9 (Cdk9) to treat cardiovascular disease by blunting cardiac hypertrophy.

### CROSS-REFERENCE TO RELATED APPLICATIONS

This application claims priority to U.S. Provisional Application No. 60/392,744 filed on Jun. 28, 2002 and 60/426,883 filed on Nov. 15, 2002, which are incorporated herein by reference in their entirety.

### STATEMENT REGARDING FEDERALLY SPONSORED RESEARCH OR DEVELOPMENT

This invention was made with government support under NIH Grant No. R01 47567 and NHLIB Grant No. HL61668 awarded by the National Institutes of Health. The United States Government may have certain rights in the invention.

### TECHNICAL FIELD

The present invention relates generally to the field of cardiology. More particularly, the present invention relates to methods of using inhibitors of cyclin dependent kinase 9 (Cdk9) to treat cardiovascular disease by blunting cardiac hypertrophy.

### BACKGROUND OF THE INVENTION

Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death. In the United States, approximately half a million individuals are diagnosed with heart failure each year, with a mortality rate approaching 50%.

Studies have shown that blunting hypertrophic growth by disrupting hypertrophic signaling pathways is beneficial to function or prognosis (Esposito et al., 2002; Sano et al., 2002). What remains unproven is which pathways and signals hold greatest potential for therapeutic benefit. In addition, signaling pathways that activate hypertrophy-associated “fetal” genes have been mapped with impressive reductionist detail (McKinsey et al., 1999; Molkentin et al., 2001), yet much less is known of mechanisms that govern hypertrophic growth itself. Even in etiologically defined genetic models of hypertropy (Molkentin et al. 1998; Adams et al., 1998; Zhang et al., 2000; Shioi et al., 2000; Bueno et al., 2000), and even where essential mediators are implicated (Minamino et al., 2002; Antos et al., 2002), the distal effectors that execute myocyte and heart enlargement remain uncertain or obscure.

One clue, involving translational control, is the activation of p70 S6 kinase, which phosphorylates the ribosomal S6 protein (Oh et al., 2001; Shioi et al., 2002). A second and separable mechanism for hypertrophic growth entails a global increase in RNA content per cell, the step that presently is least well explained. Phosphorylation of RNA polymerase II (pol II) in its carboxy-terminal domain (CTD) is a critical, essential mediator of messenger RNA production (Dahmus et al., 1996; Akhtar et al., 1996; Cho et al., 1999; Orphanides et al., 2002). In mammals, the CTD comprises 52 repeats of an evolutionally conserved serine-rich heptapeptide, Tyr-Ser-Pro-Thr-Ser-Pro-Ser. Hypophosphorylated pol II is the form recruited to promoters for transcript initiation, the CTD becomes extensively phosphorylated, primarily at Ser2 and Ser5 of the heptapeptide repeat, to overcome proximal promoter pausing and confer productive transcript elongation; dephosphorylation of the CTD recycles pol II back to the initiation-competent form Dahmus et al., 1996; Cho et al., 1999; Majello et al., 2001).

Current medical management of cardiac hypertrophy includes the use of three types of drugs: calcium channel blocking agents, β-adrenergic blocking agents, and disopyramide (Kikura and Levy, 1995). Therapeutic agents for heart failure include angiotensin II converting enzyme (ACE) inhibitors and diuretics. Other pharmaceutical agents which have been disclosed for treatment of cardiac hypertrophy include angiotensin II receptor antagonists (U.S. Pat. No. 5,604,251); and neuropeptide Y antagonists (International Patent Publication No. WO 98/33791). Despite currently available pharmaceutical compounds, prevention and treatment of cardiac hypertrophy, and subsequent heart failure, continue to present a therapeutic challenge.

Thus, there is a need for the development of new pharmacologic strategies for prophylaxis and treatment of cardiac hypertrophy in humans. In order to develop such strategies, there is a need for animal models, which accurately reflect the pathological profile of the disease, to allow identification of novel targets for therapeutic intervention.

### BRIEF SUMMARY OF THE INVENTION

The present invention relates to methods to modulate the cyclin T/Cdk9 complex and more specifically modulate Cdk9 activity to blunt the increase in ventricular mass in response to hypertrophic stimuli. The present invention is the first to describe methods of using inhibitors of Cdk9 as a treatment for heart failure.

One embodiment of the present invention is a method of treating a subject suffering from a cardiovascular disease comprising the step of administering to the subject an effective amount of a composition to modulate cyclin dependent kinase 9 (Cdk9) activity, wherein the effective amount modulates hypertrophic growth. More specifically, the cardiovascular disease of the present invention is heart failure. In specific embodiments, the composition comprises a Cdk9 inhibitor. One example of a Cdk9 inhibitor is flavopiridol.

Still further, the composition comprises a compound that modulates Cdk9 activity by prohibiting the dissociation of 7SK snRNA from cyclin T/Cdk9 complex, for example, the compound is an inhibitor of Gq. Gq inhibitors are selected from the group consisting of angiotensin II inhibitors, ACE inhibitors and endothelin inhibitors.

Still further, the composition comprises a compound that upregulates the levels of 7SK snRNA.

A further embodiment comprises a composition that is an inhibitor of calcineurin. Examples of calcineurin inhibitors are selected from the group consisting of angiotensin II inhibitors, ACE inhibitors and endothelin inhibitors.

Another embodiment of the present invention is a method of modulating myocyte enlargement in a subject at risk for cardiac hypertrophy comprising the steps of administering to the subject an effective amount of a composition to modulate cyclin dependent kinase 9 (Cdk9) activity, wherein the effective amount modulates myocyte enlargement.

Yet further, another embodiment includes a method of modulating cardiac hypertrophy comprising the step of administering to a subject an effective amount of a composition to modulate cyclin dependent kinase 9 (Cdk9) activity, wherein the effective amount modulates hypertrophic growth.

A further embodiment is a method of treating heart failure comprising the step of administering to a subject an effective amount of a composition to modulate cyclin dependent kinase 9 (Cdk9) activity. The method further comprises administering calcium channel blocking agents, β-adrenergic blocking agents, angiotensin II inhibitors or ACE inhibitors.

Another embodiment is a method of modulating a decrease in cardiac muscle contractile strength in a subject comprising the step of administering to the subject an effective amount of a composition to modulate cyclin dependent kinase 9 (Cdk9) activity, wherein the effective amount modulates the decrease in cardiac muscle contractile strength.

Still further, another embodiment of the present invention is a method of treating a subject at risk for ventricular dysfunction associated with cardiac hypertrophy comprising the steps of administering to the subject an effective amount of a composition to modulate cyclin dependent kinase 9 (Cdk9) activity, wherein the effective amount decreases ventricular dysfunction.

Yet further, another embodiment is a method of screening for a modulator of cyclin-dependent kinase 9 (Cdk9) comprising: obtaining Cdk9; contacting the Cdk9 with a candidate substance; and assaying for Cdk9 activity, wherein when the Cdk9 activity changes after the contacting, the candidate substance is a modulator of Cdk9. Specifically, the candidate substance inhibits Cdk9. In further aspects, the candidate substance prohibits the dissociation of 7SK snRNA from cyclin T/Cdk9 complex. In specific aspects of the embodiment, assaying comprises RNA hybridization, PCR, RT-PCR, or immunodetection. Immunodetection comprises Western blot, ELISA or indirect immunofluorescence.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1A-FIG. 1D shows the genetic and physiological triggers of hypertrophy active pol II CTD kinases. FIG. 1A illustrates the down-regulation of pol II phosphorylation and CTD kinase activity during normal cardiac maturation. FIG. 1B and FIG. 1C show the reactivation of pol II CTD kinases in cardiac hypertrophy. Samples were analyzed as in panel A. FIG. 1B shows αMHC-Gq (+) versus transgene-negative littermates (−). FIG. 1C shows αMHC-CaN (+) versus transgene-negative littermates (−). FIG. 1D shows partial aortic occlusion (+) versus the control surgical procedure without aortic ligation (−).

FIG. 2A-FIG. 2J show activation and function of Cdk9 in ET-1 induced cardiac myocyte hypertrophy. FIG. 2A show hyperphosphorylation of pol II and activation of CTD kinases by hypertrophic agonists. FIG. 2B shows ET-1 preferentially induces phosphorylation of the CTD repeat on Ser2, the Cdk9-dependent residue. FIG. 2C shows selective inhibition of pol II phosphorylation by DRB. FIG. 2D shows selective inhibition of Cdk9 CTD kinase activity by DRB. FIG. 2E shows selective inhibition of Cdk9 CTD kinase activity and pol II CTD phosphorylation by dominant-negative Cdk9. FIG. 2F-FIG. 2J show pharmacological and genetic inhibition implicates Cdk9 in ET-1-induced cardiac myocyte hypertrophy.

FIG. 3A-FIG. 3I show hypertrophic signals dissociate 7SK snRNA from the cyclin T/Cdk9 complex, which is sufficient to trigger cardiac muscle cell growth. FIG. 3A shows cardiac cyclin T/Cdk9 complexes contain an RNAse-sensitive inhibitor. FIG. 3B shows cardiac cyclin T/Cdk9 complexes contain 7SK snRNA (SEQ ID NO:6). FIG. 3C-FIG. 3F shows loss of Cdk9-associated 7SK RNA in (FIG. 3C) cultured cardiac myocytes treated with ET-1, (FIG. 3D) αMHC-Gq hearts, (FIG. 3E) myocardium 1 day after mechanical stress, (FIG. 3F) αMHC-CaN hearts. FIG. 3G shows recovery of cardiac cyclin T/Cdk9 complexes by a biotinylated RNA pull-down assay. FIG. 3H shows loss of 7SK snRNA suffices to activate Cdk9 in cardiac myocytes. FIG. 3I shows loss of 7SK snRNA suffices to trigger myocyte growth.

FIG. 4A-FIG. 4D show activation of Cdk9 by cyclin T1 suffices for cardiac myocyte hypertrophy in mice. FIG. 4A shows immunoblotting for cyclin T1 in low- and high-expression lines (6455, 6459) versus non-transgenic littermates (ntg). FIG. 4B shows induction of endogenous Cdk9 activity, in immune complex kinase assays. FIG. 4C shows heart size. Concentric hypertrophy is evident in the cross-section. FIG. 4D shows myocyte size.

FIGS. 5A-D show that Cdk9 activation by cyclin T1 predisposes to heart failure in concert with Gq. FIG. 5A shows in the upper and middle rows dilated cardiomyopathy. The lower row is a hematoxylin-eosin stain (bar, 20 μm). FIG. 5B shows synergistic activation of Cdk9, demonstrated by the immune complex kinase assay. FIG. 5C shows increased heart-weight-to-body-weight ratio. FIG. 5D shows rapid lethality.

FIGS. 6A-E shows Cdk9 activation by cyclin T1 predisposes to heart failure in concert with mechanical stress. FIG. 6A shows (upper row) ventricular and atrial enlargement and (lower row) hematoxylin-eosin stain (bar, 20 μm). FIG. 6B shows (top row) synergistic activation of Cdk9(immune complex kinase assay) and (lower rows) immunoblotting for pol II, cyclin T1 (endogenous plus transgenic), and secondary increases in Cdk9 and its chaperone Hsp70. Total actin is shown for comparison. FIG. 6C shows increased heart-weight-to-body-weight ratio. FIG. 6D shows increased myocyte diameter. FIG. 6E shows decreased systolic function. *, P<0.05 versus non-transgenic control littermates; †, P<0.05 versus cyclin T1 or load alone.

FIG. 7 illustrates a cluster analysis of cardiac gene expression in cyclin T1, Gq, and double-transgenic mice. Genes induced or repressed synergistically by cyclin T1 plus Gq are highlighted, for those with least effect by either transgene alone.

FIGS. 8A-F shows that catalytically inactive Cdk9 predisposes to heart failure in concert with mechanical stress. FIG. 8A shows (upper row) ventricular and atrial enlargement. And (lower row) Hematoxylin-eosin stain (bar, 20 μm). FIG. 8B shows increased heart-weight-to-body-weight ratio. FIG. 8C shows increased myocyte diameter. FIG. 8D shows decreased systolic function. FIG. 8E shows dominant-negative Cdk9 blocks Cdk9 activation by mechanical stress (immune complex kinase assay). Immunoblotting is shown for pol II, cyclin T1, and Cdk9 (endogenous plus transgenic). Total actin is shown for comparison. FIG. 8F shows decreased binding of 7SK snRNA to cyclin T/Cdk9, by RT-PCR after immunoprecipitation with antibody to cyclin T1 (Sano et al. 2002). *, P<0.05 versus non-transgenic control littermates; †, P<0.05 versus dominant-negative Cdk9 or load alone.

FIGS. 9A-E show that catalytically inactive Cdk9 predisposes to heart failure in concert with Gq. FIG. 9A shows dilated cardiomyopathy. FIG. 9B shows that dominant-negative Cdk9 blocks Cdk9 activation by Gq Top row, immune complex kinase assay. FIG. 9C shows rapid lethality. FIG. 9D shows myocyte diameter. FIG. 9E shows ystolic function.

FIGS. 10A-G shows that cardiomyocyte-restricted deletion of MAT1 causes lethal cardiomyopathy. FIG. 10A shows hearts at E14.5 to post-natal day 14. The mating strategy and nomenclature are indicated schematically at the right. FIG. 10B a-d, show ventricular and atrial enlargement at 4 weeks of age. e, f, are a hematoxylin-eosin stain, showing normal tissue structure. g, h, are a trichrome stain, indicating fibrosis. i, j, show TUNEL staining. FIG. 10C shows survival. FIG. 10D shows CTD kinase activity (immune complex assay) and expression (Western blot). FIG. 10E shows biochemical markers of apoptosis (Western blot). FIG. 10F shows transmission electron microscopy, showing mitochondrial abnormalities at 3 and 4 weeks of age (a-d and e-h, respectively). FIG. 10G shows decreased expression of mitochondrial proteins (Western blot).

FIG. 11 shows a cluster analysis of gene expression after cardiac-specific deletion of MAT1 (age=2 and 4 weeks, as shown). Genes induced or repressed by myocyte-restricted loss of MAT1 are indicated at the right.

### DETAILED DESCRIPTION OF THE INVENTION

I. Definitions

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the term “cardiovascular disease or disorder” refers to disease and disorders related to the cardiovascular or circulatory system. Cardiovascular disease and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., hypertension, arteriosclerosis, aneurysm) or veins (i.e., varicose veins, hemorrhoids). Yet further, one skill in the art recognizes that cardiovascular diseases and/or disorders can result from congenital defects, genetic defects, environmental influences (i.e., dietary influences, lifestyle, stress, etc.), and other defects or influences.

As used herein, the term “cardiac hypertrophy” refers to an enlargement of the heart due in part to an increase in the size of the myocytes. Symptoms of cardiac hypertrophy can be measured by various parameters including, but not limited to, left ventricular mass: body weight ratio; changes in cardiomyocyte size, mass, and organization; changes in cardiac gene expression; changes in cardiac function; fibroid deposition; changes in dP/dT, i.e., the rate of change of the ventricular pressure with respect to time; calcium ion flux; stroke length; and ventricular output.

As used herein, the term “cyclin” refers to a protein that accumulates continuously throughout the cell cycle.

As used herein, the term “inhibitor” refers to a compound that inhibits or blunts Cdk9 activity. It is envisioned that the inhibitor can inhibit Cdk9 activity at any point along the pathway, for example, but not limited to prohibiting dissociation of 7SK sn RNA from cyclin T/Cdk9 complex, inhibiting Gq and/or calcineurin, or inhibiting the formation of the cyclin T/Cdk9 complex.

As used herein, the term “heart failure” refers to the pathophysiological state in which the heart is unable to pump blood at a rate commensurate with the requirements of the metabolizing tissues or can do so only from an elevated filling pressure.

As used herein, the term “hypertrophic signal” indicates any stimulus, mechanical or chemical, which results in measurable symptoms of cardiac hypertrophy. Hypertrophic signals include, but are not limited to, mechanical stretch, β-adrenergic agonists, (α₁-adrenergic receptor agonists and angiotensin II.

As used herein, the term “modulator” refers to a compound that either inhibits or enhances Cdk9 activity. Specifically, the modulator inhibits or blunts Cdk9 activity.

As used herein, the term “subject” may encompass any vertebrate including but not limited to humans, mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like

As used herein, the terms “effective amount” or “therapeutically effective amount” refers to an amount that results in an improvement or remediation of the symptoms of the disease or condition.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “treating” and “treatment” as used herein refers to administering to a subject an effective amount of a the composition so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Thus, one of skill in the art realizes that a treatment may improve the disease condition, but may not be a complete cure for the disease. As used herein, the term “treatment” includes prophylaxis.

The term “palliating” a disease as used herein means that the extent and/or undesirable clinical manifestations of a disease state are lessened and/or time course of the progression is slowed or lengthened, as compared to not a substance detected by the methods of the present invention.

II. Cyclin-Dependent Kinases (Cdks)

Among the estimated 1,000 to 2,000 human protein kinases, a family of kinases activated by a family of cyclins, the cyclin-dependent kinases (Cdks), has been extensively studied because of their essential role in the regulation of cell proliferation, of neuronal and thymus functions and of transcription (Morgan, 1997; Meiger et. al., 1997; Vogt et. al., 1998 and Meijer et. al., 2000). The first identified Cdk, cdc2, was initially discovered as a gene essential for both G1/S and G2/M transitions in yeast (Nurse et. al., 1981). Following the cloning of the human cdc2 homologue, Cdk1, by complementation (Lee et. al., 1987), cdc2 homologues were found to be present in all eukaryotes from plants and unicellular organisms to humans. It was also realized that cdc2 was only the first member of a family of closely related kinases. Following the initial discovery of cyclin B in sea urchin eggs, it was also shown that cyclin B homologues were present in all eukaryotes, and that, here again, it was the first member of a large family of kinase regulators.

A. Cdks and Related Kinases: Structure

Cdks are Ser/Thr kinases (about 300 amino acids, molecular weight: 33-40 kDa) which display the eleven subdomains shared by all protein kinases. Nine Cdks and eleven cyclins have been identified in man. In addition, there are several “Cdk-related kinases” with no identified cyclin partner. These are easily recognized by their sequence homology to bona fide Cdks and by the presence of a variation of the conserved “PSTAIRE” motif, located in the cyclin-binding domain (sub-domain III) (Meyerson et. al., 1992). Until their associated cyclin is discovered (if any is associated), these “Cdk-related kinases” are named following the sequence of their PSTAIRE motif: PCTAIRE 1-3, PFTAIRE, PITAIRE, KKIALRE, PISSLRE, NKIAMRE and the PITSLRE. To be fully active, Cdk/cyclin complexes have to be phosphorylated on the residue corresponding to Cdk2 Thr160, located on the T-loop of the kinase. This phosphorylation is carried out by Cdk7/cyclin H in association with a third protein, MAT1. The Cdk subunit must also be dephosphorylated on Thr14 and Tyr15, two residues located at the border of the ATP-binding pocket.

B. Cdks and Related Kinases: Functions

(i) Cdks and Cell Cycle Control

Progression through the G1, S, G2 and M phases of the cell division cycle is directly controlled by the transient activation of various Cdks. In early to mid G1, extracellular signals modulate the activation of a first set of Cdks, Cdk4 and Cdk6 associated with D-type cyclins. Cdk4/cyclin D1 and Cdk6/cyclin D3 phosphorylate the retinoblastoma protein (pRb) and other members of the pRb family. Phosphorylation inactivates pRb, resulting in the release of the E2F and DP1 transcription factors which, in turn, control the expression of genes whose products are required for the G1/S transition and S phase progression, such as Cdk2, cyclin E and cyclin A. The Cdk2/cyclin E complex, which is responsible for the G1/S transition, also causes further phosphorylation of pRb allowing the release of an increased amount of transcription factors. During S phase, Cdk2/cyclin A phosphorylates different substrates allowing DNA replication and the inactivation of the G1 transcription factors. Around the time of the S/G2 transition, Cdk1 associates with cyclin A. Slightly later, Cdk1/cyclin B appears and triggers the G2/M transition by phosphorylating a large set of substrates such as the nuclear lamins. Phosphorylation of APC, the “Anaphase Promoting Complex”, by Cdk1/cyclin B is required for cyclin B proteolysis, transition to anaphase and completion of mitosis. These successive waves of Cdk/cyclin assemblies and activations are tightly regulated by post-translational modifications and intracellular translocations. They are coordinated and dependent on the completion of previous steps, through so-called “checkpoint” controls (Morgan, 1997; Meiger et. al., 1997; Vogt et. al., 1998 and Meijer et. al., 2000).

(ii) Cdks and Transcription

Beside their roles in controlling the cell cycle, some Cdks directly influence transcription. The Cdk7/cyclin H/MAT1 complex is a component of the TFIIH complex, a basal transcription factor. TFIIH kinase activity is responsible for phosphorylation of the C-terminal domain of the large subunit of RNA polymerase II (CTD RNA pol II), required for the elongation process.

Cdk8 associates with cyclin C and has been found in a multiprotein complex with RNA polymerase II. Like Cdk7/cyclin H, Cdk8/cyclin C phosphorylates CTD RNA pol II, but on different sites, suggesting a distinct mechanism of transcriptional regulation.

Cdk9/cyclin T is a component of the positive transcription elongation factor P-TEFb. It also displays CTD RNA pol II kinase activity.

III. Screening for Modulators

The present invention comprises methods for identifying modulators that affect the function of cyclin-dependent kinase 9 (Cdk9). These assays may comprise random screening of large libraries of candidate substances; alternatively, the assays may be used to focus on particular classes of compounds selected with an eye towards structural attributes that are believed to make them more likely to modulate the function or activity of Cdk9.

By function, it is meant that one may assay for mRNA expression, protein expression, protein activity, binding activity of cyclin-dependent kinase, or ability to associate and/or dissociate from other members of the complex, for example, cyclin T/Cdk9.

A. Modulators and Assay Formats

(i) Assay Formats

The present invention provides methods of screening for modulators of Cdk9 activity, e.g., activity of Cdk9 and/or expression of Cdk9 proteins or nucleic acids.

One embodiment, is a method of screening for modulators comprising: obtaining a Cdk9; contacting the Cdk9 with a candidate substance; and assaying for Cdk9 activity, wherein a difference between the measured activity indicates that said candidate modulator is, indeed, a modulator of the Cdk9 activity. An increase in Cdk9 activity indicates a positive modulator. A decrease in Cdk9 indicates a negative modulator.

In yet another embodiment, the assay looks at the ability of Cdk9 bind to the candidate substance to form a complex. Such methods would comprise, for example: obtaining a Cdk9; contacting the Cdk9 with a candidate substance; and determining the binding of the candidate substance to the Cdk9 wherein binding results in the formation of a complex.

Assays may be conducted in cell free systems, in isolated cells, or in organisms including transgenic animals.

(ii) Inhibitors

An inhibitor according to the present invention may be one which exerts an inhibitory effect on the expression, activity or function of Cdk9.

(iii) Candidate Substances

As used herein, the term “candidate substance” refers to any molecule that may potentially modulate Cdk9 activity, expression or function. The candidate substance may be a small molecule inhibitor, a protein or fragment thereof, or even a nucleic acid molecule or portions thereof, e.g., nucleoside analogs.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled on active, but otherwise undesirable compounds. It will be understood that an undesirable compound includes compounds that are typically toxic. These compounds have been modified to reduce the toxicity and typically have little effect with minimal toxicity and are used in combination with another compound to produce the desired effect.

On the other hand, it may prove to be the case that the most useful pharmacological compounds will be compounds that are structurally related to compounds which interact naturally with cyclin-dependent kinases. Creating and examining the action of such molecules is known as “rational drug design,” and include making predictions relating to the structure of target molecules. Thus, it is understood that the candidate substance identified by the present invention may be a small molecule inhibitor or any other compound (e.g., polypeptide or polynucleotide) that may be designed through rational drug design starting from known inhibitors of cyclin-dependent kinase activity.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule like cyclin-dependent kinase, and then design a molecule for its ability to interact with cyclin-dependent kinase. Alternatively, one could design a partially functional fragment of cyclin-dependent kinase (binding, but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Other suitable inhibitors include antisense molecules, ribozymes, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target (e.g., Cdk9) may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a Cdk9 molecule or fragment thereof is provided.

A target cyclin-dependent kinase protein may be either free in solution, fixed to a support, expressed in or on the surface of a cell. Either the target cyclin-dependent kinase protein or the compound may be labeled, thereby permitting determining of binding. In another embodiment, the assay may measure the inhibition of binding of a target cyclin-dependent kinase protein to a natural or artificial substrate or binding partner. Competitive binding assays can be performed in which one of the agents is labeled. Usually, the target cyclin-dependent kinase protein will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, cyclin-dependent kinase and washed. Bound polypeptide is detected by various methods.

Purified target, such as cyclin-dependent kinase, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g., the C-terminus of cyclin-dependent kinase) to a solid phase.

C. In Cyto Assays

Various cell lines that express cyclin-dependent kinase can be utilized for screening of candidate substances. For example, cells containing cyclin-dependent kinase with an engineered indicator can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (e.g., growth or size). Alternatively, molecular analysis may be performed in which the function of cyclin-dependent kinase and related pathways may be explored. This involves assays such as those for protein production, enzyme function, substrate utilization, mRNA expression (including differential display of whole cell or polyA RNA) and others.

D. In vivo Assays

The present invention particularly contemplates the use of various animal models. For example, transgenic animals may be created with constructs that permit cyclin-dependent kinase expression and activity to be controlled and monitored. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), and U.S. Pat. No. 6,201,165, the teachings of which are generally known and are incorporated herein.

Treatment of animals with test compounds (e.g., Cdk9 inhibitors) involve the administration of the compound, in an appropriate form, to the animal. Administration is by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

E. Production of Inhibitors

In an extension of any of the previously described screening assays, the present invention also provide for methods of producing inhibitors. The methods comprising any of the preceding screening steps followed by an additional step of “producing the candidate substance identified as a modulator of” the screened activity.

IV. Compositions

The present invention provides a composition comprising the inhibitors and/or modulators of the present invention and a pharmaceutical carrier. The compositions of the present invention are used to treat cardiovascular diseases, including, but not limited to, coronary heart disease, arteriosclerosis, ischemic heart disease, angina pectoris, myocardial infarction, heart failure and other diseases of the arteries, arterioles and capillaries or related complaint. Accordingly, the invention involves the administration of composition as a treatment or prevention of any one or more of these conditions or other conditions involving hypertrophy of myocytes or increases in ventricular mass, as well as compositions for such treatment or prevention.

The compositions disclosed herein are administered via injection, including, but not limited to subcutaneous or parenteral including intravenous, intraarterial, intramuscular, intraperitoneal, intramyocardial, transendocardial, transepicardial, intranasal administration as well as intrathecal, and infusion techniques.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Moreover, for human administration, preparations should meet sterility, pyrogenicity, general safety and purity standards as required by FDA Office of Biologics standards. The formulations are easily administered in a variety of dosage forms such as injectable solutions, drug release capsules and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

V. Treatment of Cardiovascular Disease

A. Treatment

Embodiments of the present invention relate to methods of treating cardiovascular disease. The methods comprise modulating the cyclin T/Cdk complex and more specifically modulating Cdk9 activity to blunt the increase in ventricular mass in response to hypertrophic stimuli.

Cardiovascular diseases and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., hypertension, arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), myocardial infarction and ischemic heart disease.

In specific embodiments, the present invention comprises a method of treating a subject suffering from a cardiovascular disease comprising the step of administering to the subject an effective amount of a composition to modulate cyclin dependent kinase 9 (Cdk9) activity, wherein the effective amount modulates hypertrophic growth. It is evisioned that the composition is a pharmaceutical composition that comprises a Cdk9 inhibitor. Specifically, the Cdk9 inhibitor is flavopiridol. Yet further, it is envisioned that derivatives of flavopiridol may also be used.

In further embodiments, the composition comprises a compound that modulates Cdk9 activity by prohibiting the dissociation of 7SK snRNA from cyclin T/Cdk9 complex. It is envisioned that by prohibiting the dissociation of 7SK snRNA from cyclin T/Cdk9 complex it will inhibit Cdk9 activity resulting in blunting or a decrease in hypertrophic growth, i.e. ventricular mass, myocyte enlargement, etc.

Specific compounds that are used to prohibit and/or prevent dissociation of 7SK snRNA from cyclin T/Cdk9 complex include, but are not limited to inhibitors of Gq and calcineurin. Such inhibitors of Gq and calcineurin include, but are not limited to angiotensin II inhibitors, ACE inhibitors and endothelin inhibitors and derivatives thereof.

Yet further, other compounds can be used to modulate Cdk9 activity, for example, a compound that upregulates the levels of 7SK snRNA. Upregulation of the levels of 7SK snRNA can provide sufficient amounts of 7SK snRNA to ensure that 7SK snRNA stays associated with the cyclin T/Cdk9 complex.

Accordingly, the invention involves the composition of the present invention as a treatment or prevention of any one or more of these conditions or other conditions involving heart disease, more specifically cardiac hypertrophy, as well as compositions for such treatment or prevention.

Cardiac hypertrophy in response to an increased workload imposed on the heart is a fundamental adaptive mechanism. It is a specialized process reflecting a quantitative increase in cell size and mass (rather than cell number) as the result of any or a combination of neural, endocrine or mechanical stimuli. Thus, this adaptive mechanism permits the heart to compensate for overloading and plays a significant role in augmenting the contractile strength of the myocytes, i.e., cardiac muscle.

Another embodiment is a method of modulating a decrease in cardiac muscle contractile strength in a subject comprising the step of administering to the subject an effective amount of a composition to modulate cyclin dependent kinase 9 (Cdk9) activity, wherein the effective amount modulates the decrease in cardiac muscle contractile strength.

It is known and understood by those of skill in the art that stroke volume or ventricular work is related to the level of venous inflow, as measured by atrial pressure, or by ventricular end-diastolic volume or end-diastolic pressure. Thus, in a normal heart, the heart will pump whatever volume is brought to it by the venous circulation. The increase in contractile force that occurs in response to ventricular dilation is related to the myofibrillar organization, for example stretching of the sarcomeres. In cardiac hypertrophy, the adaptive alterations in the myocyte structure and function result in a decrease in the work of the heart, stroke volume, despite the increase in atrial pressure, thus the heart has decreased contractile strength resulting in ventricular dysfunction ultimately leading to heart failure. Contractile strength or contractility can be measured by measuring the maximum rate of change in pressure (dp/dt max). Clinically, contractility is measured by ejection fraction. Normally, the heart ejects about 60% of its volume each beat, thus a decrease in the volume is an indicator of decreased contractility or contractile strength and ventricular dysfunction.

Still further, the present invention comprises a method of treating a subject at risk for ventricular dysfunction associated with cardiac hypertrophy comprising the steps of administering to the subject an effective amount of a composition to modulate cyclin dependent kinase 9 (Cdk9) activity, wherein the effective amount decreases ventricular dysfunction.

Yet further, the methods comprise administering to a subject in need thereof an amount of a substance effective to diminish or reverse progression of the dysfunction. In the context of prophylaxis, a subject in need thereof includes, but is not limited to, individuals in the general population who are 55 years of age and older; individuals who have a genetic predisposition to developing cardiac hypertrophy; dilated cardiac myopathy patients; hypertensive patients; patients with renal failure and vascular hypertension; individuals with vascular hypertensive due to pressure overload, volume overload, or increased peripheral bed resistance; individuals with respiratory ailments such as emphysema or cystic fibrosis; chronic asthmatics; individuals with tuberculosis; and organ transplant patients.

The present invention also comprises a method of modulating myocyte enlargement in a subject at risk for cardiac hypertrophy comprising the steps of administering to the subject an effective amount of a composition to modulate cyclin dependent kinase 9 (Cdk9) activity, wherein the effective amount modulates myocyte enlargement. Thus, it is envisioned that the composition modulates Cdk9 activity to blunt enlargement of myocytes in vitro or in vivo.

B. Administration and Treatment Regimens

It is envisioned one of skill in the art will know the most advantageous routes of administration depending upon the disease. In specific embodiments, it is contemplated that composition can be administered via injection, which includes, but is not limited to subcutaneous, intravenous, intraarterial, intramuscular, intraperitoneal, intramyocaridal, transendocardial, transepicardial, intranasal and intrathecal.

Yet further, it is envisioned that composition of the present invention can be administered to the subject in an injectable formulation containing any compatible carrier, such as various vehicles, adjuvants, additives, and diluents. Yet further, the composition can be administered parenterally to the subject in the form of slow-release subcutaneous implants or targeted delivery systems such as monoclonal antibodies, iontophoretic, polymer matrices, liposomes, and microspheres.

Treatment regimens may vary as well, and often depend on the cardiovascular disease or disorder, disease progression, and health and age of the subject. Obviously, certain types of cardiovascular disease will require more aggressive treatment, while at the same time, certain patients cannot tolerate more taxing protocols. The clinician will be best suited to make such decisions based on the known efficacy and toxicity (if any) of the therapeutic formulations.

Suitable regimes for initial administration and further doses or for sequential administrations also are variable, and may include an initial administration followed by subsequent administrations; but nonetheless, may be ascertained by the clinician.

For example, the composition of the present invention can be administered initially, and thereafter maintained by further administration. For instance, a composition of the invention can be administered in one type of composition and thereafter further administered in a different or the same type of composition. For example, a composition of the invention can be administered by intravenous injection to bring blood levels to a suitable level. The subject's levels are then maintained by a subcutaneous implant form, although other forms of administration, dependent upon the subject's condition, can be used.

The effective amount is an amount of the composition of the present invention that blunt or reduce hypertrophic growth, or decrease ventricular mass, prevent an increase in ventricular mass, and/or reduce myocyte hypertrophic growth. Thus, an effective amount is an amount sufficient to detectably and repeatedly ameliorate, reduce, minimize or limit the extent of the disease or its symptoms.

Symptoms of cardiac hypertrophy can be measured by various parameters including, but not limited to, left ventricular mass: body weight ratio; changes in cardiomyocyte size, mass, and organization; changes in cardiac gene expression; changes in cardiac function; fibroid deposition; changes in dP/dT, i.e., the rate of change of the ventricular pressure with respect to time; calcium ion flux; stroke length; and ventricular output. Thus, an effective amount of the composition of the present invention is an amount sufficient to detectably and repeatedly ameliorate, reduce, minimize or limit the extent of the these symptoms.

Yet further, an effective amount of the composition of the present invention is an amount sufficient to detectably and repeatedly ameliorate, reduce, minimize or limit the extent of the any biochemical alteration associated with cardiac hypertrophy. Such biochemical alterations include, but at not limited to, decreases in norepinephrine stores, decreases in β-adrenergic receptors, decreases in calcium uptake by the sarcoplasmic reticulum, decreases in calcium efflux from the sarcoplasmic reticulm, increases in calcium channels and increase in calcium influx.

The precise determination of what would be considered an effective dose may be based on factors individual to each subject, including their size, age, size of the ventricular mass, and amount of time since hypertrophic growth. Therefore, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Of course, for any composition to be administered to an animal or human, and for any particular method of administration, it is preferred to determine the toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The treatments may include various “unit doses.” Unit dose is defined as containing a predetermined-quantity of the composition. The quantity to be administered, and the particular route and formulation, are within the skill of those in the clinical arts. A unit dose need not be administered as a single injection but may comprise continuous infusion over a set period of time.

VI. Combined Cardiovascular Disease Treatments

In order to increase the effectiveness of the composition, it may be desirable to combine these compositions and methods of the invention with a known agent effective in the treatment of cardiovascular disease or disorder, for example known agents to treat heart failure. In some embodiments, it is contemplated that a conventional therapy or agent, including but not limited to, a pharmacological therapeutic agent, a surgical therapeutic agent (e.g., a surgical procedure) or a combination thereof, may be combined with the composition of the present invention.

This process may involve contacting the cell(s) with an agent(s) and the composition of the present invention at the same time or within a period of time wherein separate administration of the agent and the composition to a cell, tissue or organism produces a desired therapeutic benefit. The terms “contacted” and “exposed,” when applied to a cell, tissue or organism, are used herein to describe the process by which the composition and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both the composition and one or more agents, or by contacting the cell with two or more distinct compositions or formulations, wherein one composition includes the composition and the other includes one or more agents.

The treatment may precede, be co-current with and/or follow the other agent(s) by intervals ranging from minutes to weeks. In embodiments where the composition, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) with the composition. In other aspects, one or more agents may be administered within of from substantially simultaneously, about minutes to hours to days to weeks and any range derivable therein, prior to and/or after administering the composition.

Administration of the composition to a cell, tissue or organism may follow general protocols for the administration of cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, or a vasopressor. Other drug therapies include treatment agents for congestive heart failure, for example, but not limited to calcium channel blocking agents, β-adrenergic blocking agents, angiotensin II inhibitors or ACE inhibitors. ACE inhibitors include drugs designated by the trademarks Accupril®, Altace®, Capoten®, Lotensin®, Monopril®, Prinivil®, Vasotec®, and Zestril®.

B. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Western Blotting and CTD Kinase Assays

Immune complex kinase assays were performed as described (Yang et al., 1996), using polyclonal rabbit antibody against Cdk7 or Cdk9 together with protein A-Sepharose. Kinase assays were performed by adding 25 μt of 50 mM Tris-HCl [pH 7.4], 10 mM MgCl₂, 5 mM dithiothreitol, 2.5 mM MnCl₂, 5 μM ATP, 5 μCi of [γ-³²P]ATP, and 200 ng of GST-CTD to the beads and incubating at room temperature for 60 min. The complexes were resolved by SDS-polyacrylamide gel electrophoresis, and CTD phosphorylation was visualized by autoradiography and quantified using a phosphorimager and ImageQuant version 5.2 (Molecular Dynamics). Where indicated, immunoprecipiates were incubated with 50 μg/ml of bovine pancreas ribonuclease A (RNase A; Sigma) for 60 min prior to the CTD kinase assay.

Western blotting was performed using the identical cell lysates. The following antibodies were used: rabbit antibodies against pol II, Cdk7, Cyclin H, MAT1, CyclinT1, Hsp70, Hsp90 and Cdc37; goat antibody against Cyclin T2a/b; and mouse monoclonal antibodies to the Ser2 and Ser5 CTD phoshopeptides (Patturajan et al., 1998; Herrmann et al., 2001). Protein expression was visualized using horseradish peroxidase-conjugated second antibodies and enhanced chemiluminescence reagents (Amersham Pharmacia Biotech).

### Example 2

### Mouse Models of Cardiac Hypertrophy

Pressure-overload cardiac hypertrophy was induced in 12-week-old adult FVB mice (Harlan Laboratories) by transverse aortic banding, between the right innominate artery and left carotid artery (Zhang et al., 2000). The surgical intervention was validated by Doppler echocardiography, and only mice were used in which the left-to-right carotid flow velocity ratio was 0.35:1 or less. As the control, a “sham” operation was performed on age-matched animals, including anesthesia, intubation, thoracotomy, and manipulation of the aorta without occlusion. Cardiac-specific Gαq (Molkentin et al., 2001) and calcineurin (Adams et al., 1998) transgenic mice were used. Transgene-negative wild-type littermates were used as the control.

A cardiac-specific cyclin T1 transgene was constructed using the mouse cyclin T1 coding sequence (Kwak et al., 1999), cloned 3′ to the 5.5-kbp mouse α-MHC promoter (Subramaniam et al., 1991) and 5′ to the human growth hormone polyadenylation sequence. Expression cassettes were released with BamHI, and microinjected into the pronuclei of fertilized FVB oocytes.

### Example 3

### Recombinant Adenoviruses and Cell Culture

pAd-Easyl and pAd-TrackCMV were used for cell culture (He et al., 1998). Catalytically inactive, dominant-negative Cdk7 (dn Cdk7; K41N/K42Q) (Matzuoka et al., 2000) was generated by a two-step PCR method, using the wild-type mouse cDNA as template. Catalytically inactive, dominant-negative human Cdk9 (dn Cdk9; D167N) was detailed previously by de Falco et al. (2000). The cDNAs were subcloned into pAd-, for co-expression of eGFP, and the vectors subjected to homologous recombination in bacteria with pAd-Easy 1. Viruses were propagated on 293 cells and purified by CsCl₂ banding followed by dialysis.

Neonatal ventricular myocytes from 1 to 2 day-old Sprague-Dawley rats were subjected to Percoll gradient centrifugation and differential plating, to enrich for cardiac myocytes and deplete nonmyocytes (Oh et al., 2001). Cells were infected for 6 hr, after overnight culture, at a multiplicity of infection (MOI) of 20, then were cultured in serum-free medium for an additional 24 hr before the addition of agonists. The efficiency of viral gene transfer is >95% under the conditions used. DRB (Sigma) was dissolved in dimethylsulfoxide (DMSO) and then in culture medium to the desired final concentration in 0.1% (vol/vol) DMSO.

Cells were incubated for 24 hr with 1 μCi/ml of either precursor, in the absence or presence of ET-1 and [³H]uridine and [³H]phenylalanine incorporation was measured. Total RNA was extracted using TRIzol (Invitrogen), for simultaneous extraction of DNA from the samples.

Myocyte apoptosis was monitored by flow cytometry as hypodiploid DNA, using FITC-conjugated MF20 antibody to sarcomeric myosin heavy chains and propidium iodide in the presence of RNase A (Oh et al., 2001).

The sequences used for antisense phosphorothioate oligonucleotides (Molecula Research Laboratories) were: antisense 7SK, (SEQ. ID. NO. 1) 5′-CCTTGAGAGCTTGTTTGG AGG-3′; antisense eGFP, (SEQ. ID. NO.2) 5′-CGTTTACGTCGCCGTCCAGC-3′. Anti sense oligonucleotides were transfected using Effecten (Qiagen) into cardiac myocytes expressing eGFP. RNA expression, Cdk9 kinase activity, and [³H]uridine incorporation were determined 48 hr later.

### Example 4

### Co-Precipitation of P-TEFb and 7SK snRNA

Total RNA was isolated from Cdk9, cyclin T1, cyclin T2, and Cdk7 immune complexes using TRIzol and subjected to DNase I (Invitrogen) for 15 min at room temperature, followed by reverse transcription using antisense oligonucleotides for 7SK snRNA. The PCR primers were: sense, (SEQ. ID. NO. 3) GGATGTGAGGCGATCTGGCTG; antisense, (SEQ. ID. NO. 4) TAAAGAAAGGCAGACTGCCAC. PCR products were subjected to electrophoresis on 2% agarose gel and stained with ethidium bromide.

For the RNA pull-down assay (Yang et al., 2001), lysates were incubated with 1.8 μM biotinylated antisense 2′-OMe RNA oligonucleotide ((SEQ. ID. NO. 5) 5′-biotin-ACCUUGAGAGCUUGUUUGGAGG-3′, complementary to nucleotides 221-241), for 1 hr at room temperature in the presence of 0.2 U/mL RNasin, then with streptavidin-agarose (Sigma) for 1 hr at 4° C. Beads were recovered by centrifugation and washed 5 times, and associated cyclin T1/Cdk9 was demonstrated by Western blot analysis.

### Example 5

### Genetic and Physiological Triggers of Hypertrophy Activate pol II CTD Kinases

Western blotting was performed using antibody that recognizes both hyperphosphorylated and hypophosphorylated pol II (IIo and IIa, respectively) as described in Example 1. Pol II phosphorylation and the principal CTD kinases during normal cardiac maturation were monitored.

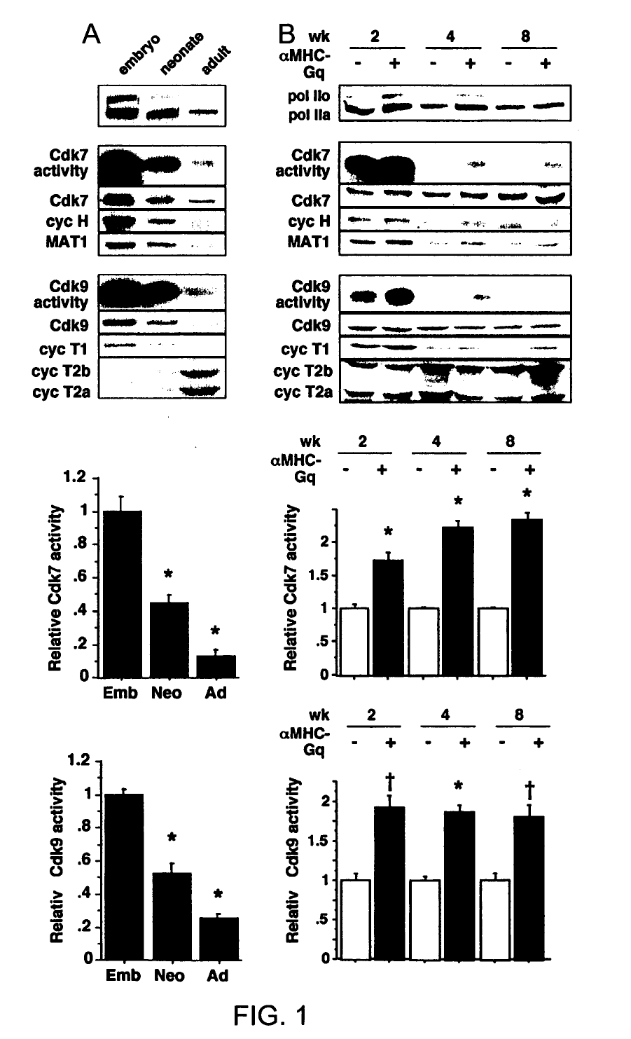

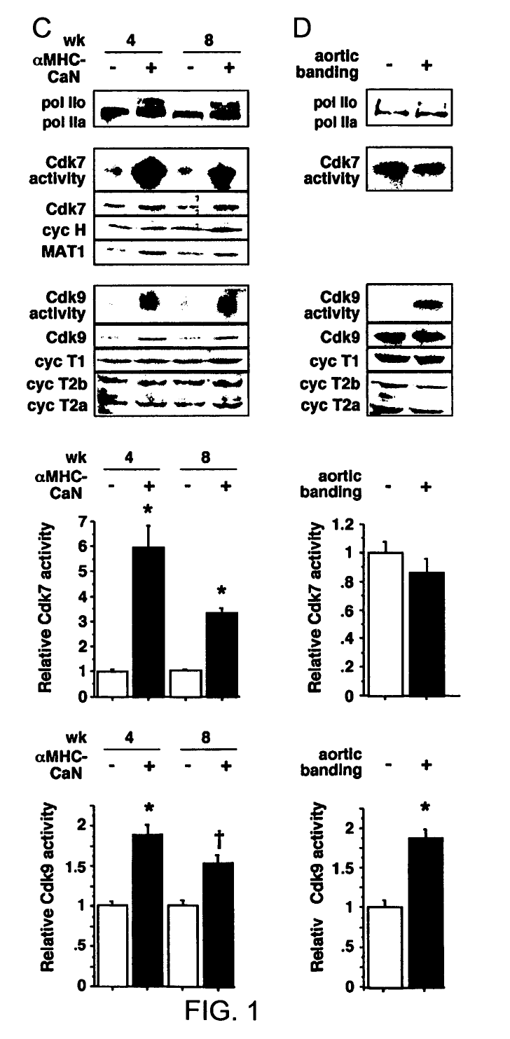

Although both forms were most highly expressed in embryonic hearts and decreased after birth, the proportion of pol IIo, the form required for productive transcript elongation, versus total pol II, decreased from 31±2% in embryonic myocardium to 4±0.2% in adult hearts (n=5; P<0.0001; FIG. 1A).

Immune complex kinase assays were performed using GST-CTD as substrate to test the prediction that this shift entailed down-regulation of one or more CTD kinase activities. Both Cdk7 and Cdk9 activity were readily detected in embryonic hearts, accompanied by expression of cyclin H, Cdk7, MAT1 (ménage-a-trois, the third constituent of CAK), cyclin T1, and Cdk9. By contrast, both CTD kinase activities, expression of both Cdks, and expression of the respective cyclins, each decreased at least 75% after birth. In addition to cyclin T1, Cdk9 was regulated by cyclin T2, which existed as two alternatively spliced isoforms. Unlike any of the aforementioned proteins, cyclin T2a and T2b both increased during cardiac maturation, in agreement with their up-regulation in differentiated skeletal muscle (De Luca et al., 2001); little is known concerning the potential function of T2 cyclins (Kwak et al., 1999; Wimmer et al., 1999; Peng et al., 1998).

The frequent reversion to “fetal” protein levels in cardiac hypertrophy provided a rationale to test for reexpression of CTD kinases or their cyclins in this setting. Reinduction of the kinases' activity independently of protein expression was a related possibility, seen, for example, with transforming growth factor beta-activated kinase in hypertrophic myocardium (Zhang et al., 2000). Three complementary mouse models of cardiac hypertrophy were analyzed by Western blotting as in Example 1. Thus, in transgenic mice with hypertrophy triggered by the signaling protein Goαq or calcineurin, pot IIo increased up to 2-fold, compared with wild-type non-transgenic littermates at each age examined, with little or no change in pot IIa (FIGS. 1B, C). Consequently, the proportion of IIo increased from 16% in control mice of both lines to ˜30% in transgene-positive ones.

In accordance with the enhanced phosphorylation of pot II, Cdk7-dependent and Cdk9-dependent CTD kinase activities both increased in the myocardium. As previously reported for Cdk7 a week after aortic banding (Abdellatif et al., 1998), gain-of-function mutations for Gαq and calcineurin induced small but reproducible increases in one or more components of the Cdk7/cyclinH/MAT1 complex. By contrast to the activation of Cdk7 in these transgenic models and in chronic mechanical load (partial aortic constriction for 3 wk, acute mechanical load provoked only the activation of Cdk9 (FIG. 1D). Because this acute induction of Cdk9 kinase activity precedes by days the hypertrophic growth that occurs after aortic banding, hyperphosphorylation of the CTD and the activation of Cdk9 cannot be a consequence of cell enlargement. Interestingly, no increase occurred—in any of the three models—in expression of Cdk9 or its cyclins, to explain the observed increases in Cdk9 activity.

Thus, both CTD kinases were activated by the genetic signals for cardiac growth and by chronic workload, indicating that the induction of CTD kinase activity is common to all models tested. However, activation of Cdk9, specifically, occurred with acute aortic banding.

### Example 6

### Cdk9 is the Essential Pol II CTD Kinase for Hypertrophic Growth

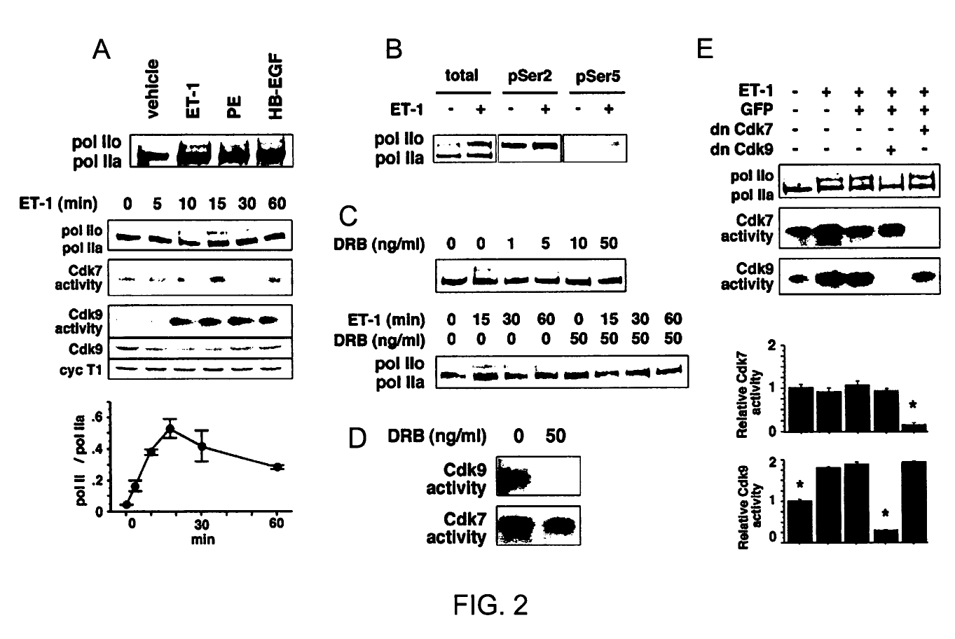

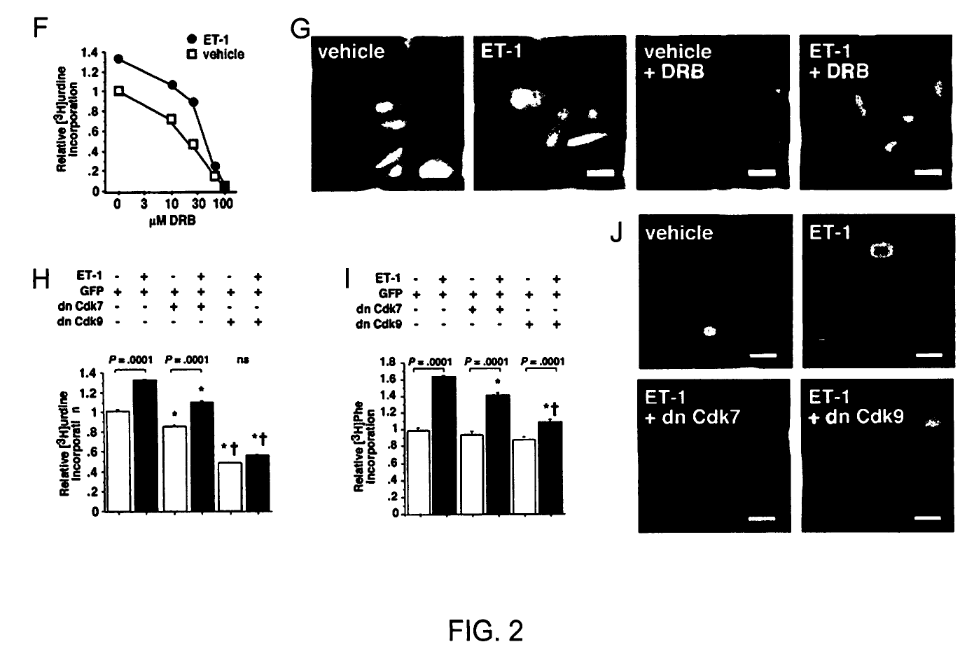

Cultured cardiac myocytes were subjected to three extracellular signals for hypertrophy. Hyperphosphorylation of pol II was induced by all agonists tested: endothelin-1 (ET-1) and the α₁-adrenergic ligand phenylephrine (PE), both of which signal through Gαq (Dorn et al., 1999; Doi et al., 1999) and calcineurin (Zhu et al., 2000; Taigen et al., 2000), as well as heparin-binding epidermal growth factor (HB-EGF), a member of the EGF family that was recently shown to be a secreted mediator of diverse growth signals in the heart (Asakura et al., 2002) (FIG. 2A).

Briefly, ventricular myocytes were analyzed by Western blotting as in Example 1. Ventricular myocytes were serum-starved for 24 hr, stimulated for 15 min with ET-1 (0.1 μM), PE (0.1 μM), HB-EGF (1 nM), or the vehicle, and analyzed by Western blotting with antibody to the pol II CTD. Ventricular myocytes were stimulated with ET-1 for the indicated times.

Pol II phosphorylation was detected within 10 min of stimulation, and peaked at 15 min; the proportion of pol IIo increased 9-fold, from 4% at baseline to a maximum of 36%. As was true for acute pressure overload in vivo, only Cdk9-associated CTD kinase activity was increased, without parallel change in Cdk7 activity (FIG. 2A, below). No increase was detected in levels of Cdk9 or cyclin T1. Likewise, no change was seen in levels of cyclins T2a and T2b.

Ser2 and Ser5 of the CTD heptad repeat are the preferred substrates of Cdk9 and Cdk7, respectively (Zhou et al., 2000). Using antibodies specific to each of these phospho-epitopes (Patturajan et al., 1998; Herrmann et al., 2001), ET-1 was shown to preferentially induce phosphorylation at Ser2 (FIG. 2B). To establish more directly which CTD kinase(s) mediate ET-1-induced phosphorylation of pol II, a pharmacological inhibitor of Cdk9 and dominant-inhibitory proteins were employed. The nucleoside analog 5,6-dichloro-1-beta-D-ribofuranosylbenzimidazole (DRB) specifically inhibits transcript elongation by pol II, not transcript initiation, and has selective activity against Cdk9 (Zhu et al., 1997).

Next, cells were preincubated with DRB for 2 hr at the concentrations shown, and were assayed for pol II phosphorylation after stimulation with ET-1 for 15 min or the indicated intervals. Immune complexes were treated in vitro with 50 μM DRB and assayed by phosphorylation of the recombinant GST-CTD substrate. In cultured cardiac myocytes, 50 μM DRB inhibited ET-1-induced pol II phosphorylation (FIG. 2C), without impairing the phosphorylation of mitogen-activated protein kinases that also are downstream targets of ET-1. Based on this effect, 50 μM DRB was added in vitro to cardiac Cdk9 and Cdk7 immune complexes, and assayed for its effect on phosphorylation of the recombinant CTD substrate. DRB blocked Cdk9 activity almost completely, with little or no effect on Cdk7 (FIG. 2D).

Next, cells were infected with adenoviruses shown, and were assayed 15 min after stimulation with ET-1. Adenovirus-mediated delivery of catalytically inactive, dominant-negative Cdk9 (dn Cdk9) specifically inhibited Cdk9 CTD kinase activity, with no measurable effect on Cdk7 (FIG. 2E). Conversely, dominant-negative Cdk7 suppressed Cdk7 activity, with no effect on Cdk9. In agreement with the pharmacological evidence implicating Cdk9 not Cdk7 in ET-1-stimulated cells, the phosphorylation of endogenous pol II was blocked only by dominant-negative Cdk9 and not by dominant-negative Cdk7. Thus, rapid activation of Cdk9 occurs in response to ET-1, and is essential for ET-1-induced pol II phosphorylation.

Next, cells were pretreated with 50 ng/ml DRB (F, G) or recombinant adenovirus expressing eGFP to delineate infected cells, with and without catalytically inactive CTD kinases as dominant-interfering proteins (FIG. 2H-FIG. 2J). Hypertrophy was monitored as [³H]uridine incorporation (FIG. 2F, FIG. 2H; n=4 per group) and [³H]phenylalanine incorporation (FIG. 2I; n=6 per group), corrected for DNA content. Myocyte size was visualized with eGFP (FIG. 2G, FIG. 2J). Bar, 100 μm.

Because Cdk9 is the CTD kinase common to all hypertrophic stimuli tested (acute as well as chronic) and, moreover, is required for CTD phosphorylation in ET-1-induced hypertrophy, this predicts that Cdk9, not Cdk7, would be essential for hypertrophic growth induced by ET-1. DRB induced significant inhibition of both basal and ET-1 induced RNA synthesis in cardiac myocytes, with ˜80% inhibition of each at a concentration of 50 μM (FIG. 2F). As expected from these findings, DRB blocked the associated increase in myocyte size (FIG. 2G).

Catalytically inactive Cdk9 and Cdk7 was expressed by viral gene transfer, and [³H]-uridine incorporation into RNA was monitored, with and without ET-1 stimulation (FIG. 2H) to establish whether genetic inhibition of one or both CTD kinases would block RNA synthesis in cardiac myocytes. ET-1 augmented [³H]uridine incorporation by one-third in control (enhanced GFP-infected) myocytes. Dominant-negative Cdk7 reduced [³H]uridine incorporation by ˜16%, either in the absence or presence of ET-1, and thus had no effect on the extent of induction by agonist. Dominant-negative Cdk9 inhibited [³H]uridine incorporation by more than 50% in the absence or presence of ET-1, with no significant induction by ET-1 remaining. Likewise, dominant-negative Cdk9 had the greater effect on [³H]phenylalanine incorporation (FIG. 2I). In agreement with these results, dominant-negative Cdk9 markedly inhibited ET-1-induced cardiac myocyte enlargement, whereas dominant-negative Cdk7 did not (FIG. 2J). Because both kinase mutations were expressed in bicistronic vectors coexpressing eGFP, allowing equal infectivity to be ensured, and because the dominant-interfering effect of each inactive protein was confirmed, these functional disparities cannot be ascribed to trivial technical differences in gene delivery or failure to suppress Cdk7 equivalently. Thus, together with pharmacological inhibition, the results of viral gene transfer suggested that Cdk9 was an essential pol II kinase for cardiac hypertrophic growth.

Under conditions of each inhibitor sufficient to block the induction of pol II phosphorylation by ET-1 almost completely, no increase in apoptosis occurred. Higher concentrations of DRB that block RNA synthesis more completely did induce cell death.

### Example 7

### A Cdk9 Inhibitor, from the Cyclin T-Cdk9 Complex

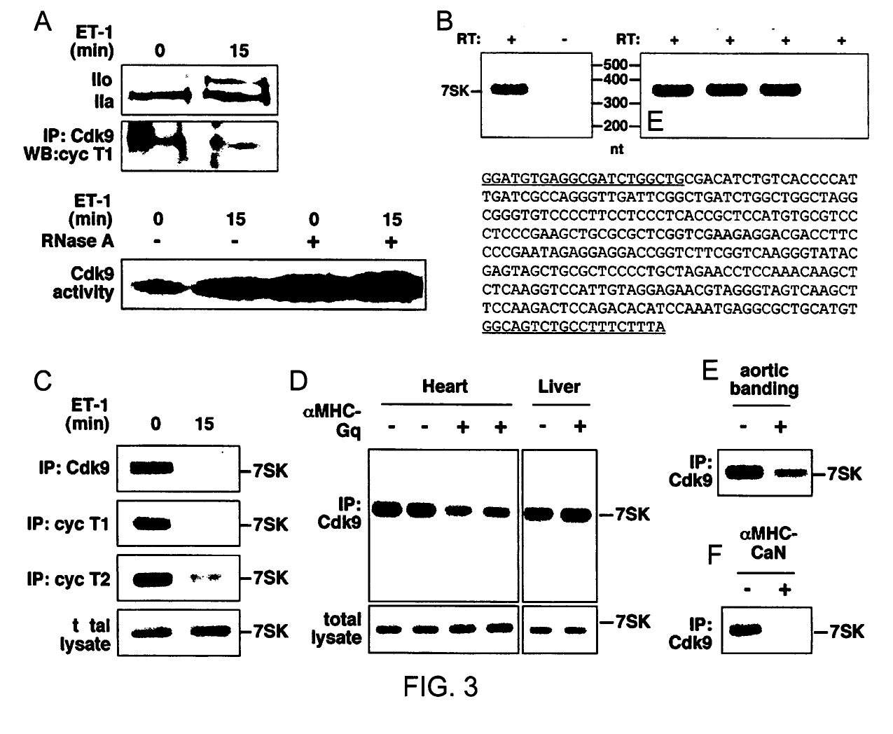

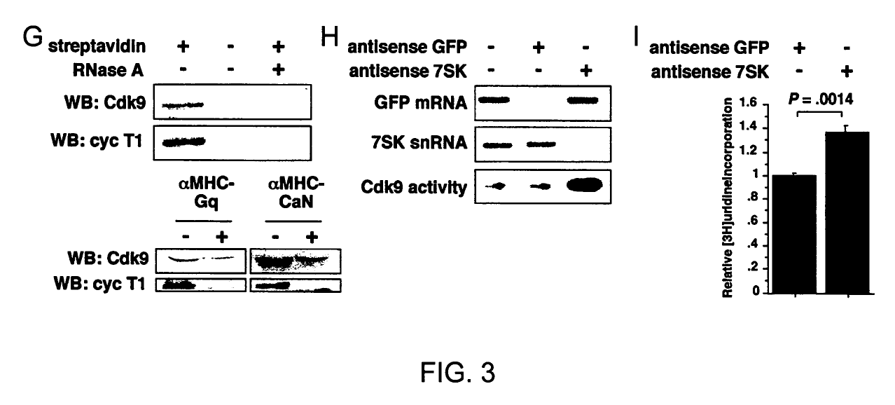

Briefly, ventricular myocytes were stimulated with ET-1 for 15 min, which increased pol II CTD phosphorylation (top) and Cdk9 CTD kinase activity (bottom). Under these conditions, no change occurred in cyclin T1-associated Cdk9 (middle). After treatment with RNase A, immune complex CTD kinase activity was even greater than in agonist-stimulated cells. No significant increase was observed, in any model, in levels of Cdk9 or its activators, the T cyclins. Furthermore, no increase occurred in the assembly of cyclinT/Cdk9 in same acute cell culture experiments where both Cdk9 kinase activity and Cdk9-dependent phosphorylation of endogenous pol II were increased (FIG. 3A).

Recently, two independent groups identified 7SK, a small nuclear RNA of previously unknown function, as a component of the cyclin T/Cdk9 complex (Yang et al., 2001; Nguyen et al., 2001). 7SK suppresses Cdk9 kinase activity, inhibits productive transcript elongation, and is dissociated from the cyclin T/Cdk9 complex by treatments that enhance pol II phosphorylation and transcription (Yang et al., 2001; Nguyen et al., 2001). As a first step to test for this inhibitory RNA in endogenous cardiac cyclin T/Cdk9 complexes, Cdk9 immune complexes were treated with RNase A, then assayed for activity towards the recombinant GST-CTD peptide. RNase A increased Cdk9 kinase activity in control cells to levels even higher than did ET-1 (FIG. 3A).

Next, RNA was isolated from immune complexes following precipitation with each of the four antibodies shown, and was subjected to RT-PCR using primers specific for 7SK RNA. The RT-PCR product was cloned and sequence-verified; only one nucleotide difference was seen from the published human sequence. The PCR primers used are underlined.

The association of 7SK with cardiac cyclin T/Cdk9 complexes was substantiated by amplification of this snRNA contingent on the presence of reverse transcriptase, regardless of whether antibody to Cdk9, cyclinT1, or cyclin T2 was used, and its identity was verified by cDNA sequencing (FIG. 3B). No 7SK RNA was associated with Cdk7, and no PCR product was formed in the absence of reverse transcriptase.

7SK physically associated with cyclin T/Cdk9 was assayed in cardiac myocytes with and without prior ET-1 treatment (FIG. 3C) in order to determine whether hypertrophic stimuli resulted in the release of 7SK from cardiac P-TEFb. ET-1 caused the rapid loss of Cdk9-associated 7SK snRNA, within 15 min. These findings were substantiated using each of three antibodies to the cyclin T/Cdk9 complex. Levels of 7SK RNA in the corresponding total cell lysates were unchanged. Results equivalent to those with ET-1 were observed in the myocardium of Gq transgenic mice—that is, loss of 7SK RNA specifically from the cyclin T/Cdk9 complex, with no change in 7SK RNA expression (FIG. 3D). As expected, the association of 7SK RNA with Cdk9 was unchanged in liver, which does not express the cardiac-specific Gq transgene. Thus, hypertrophic signals in cultured cardiac myocytes and the intact heart induce the dissociation of 7SK RNA from endogenous cyclin T/Cdk9. Similar results were seen with acute mechanical stress (FIG. 3E) and hypertrophy induced by calcineurin (FIG. 3F). Conversely, endogenous cyclin T/Cdk9 was recovered from cardiac lysates using streptavidin-agarose plus a biotinylated 2′-O-methyl (2′-OMe) oligonucleotide complementary to 7SK snRNA (FIG. 3G): this method confirmed independently the dissociation of 7SK snRNA from cardiac cyclin T/Cdk9 for Gq and calcineurin.

Antisense oligonucleotides were used because the pivotal target was snRNA (not a protein), and insufficient information existed to interfere specifically with just its binding to the complex. Potential factitious effects of oligonucleotides including formation of the RNA-DNA duplex and activation of RNase H (Braasch et al., 2002) were controlled using antisense knockdown of GFP. Loss of 7SK snRNA markedly increased endogenous Cdk9 activity, and induced as large an increase in uridine incorporation as did ET-1 itself (FIGS. 3H, I; FIG. 2H).

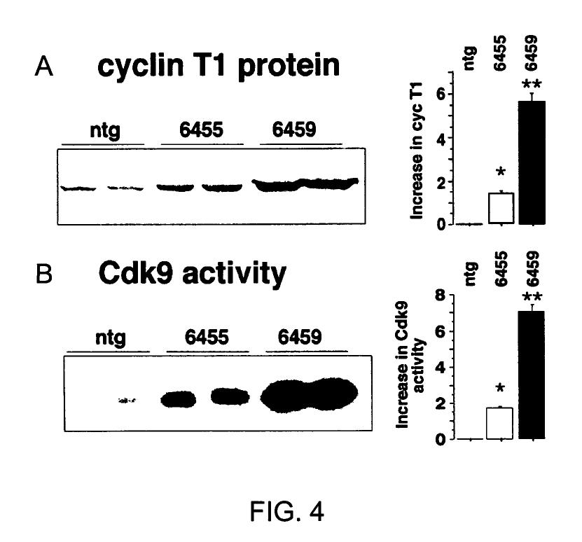

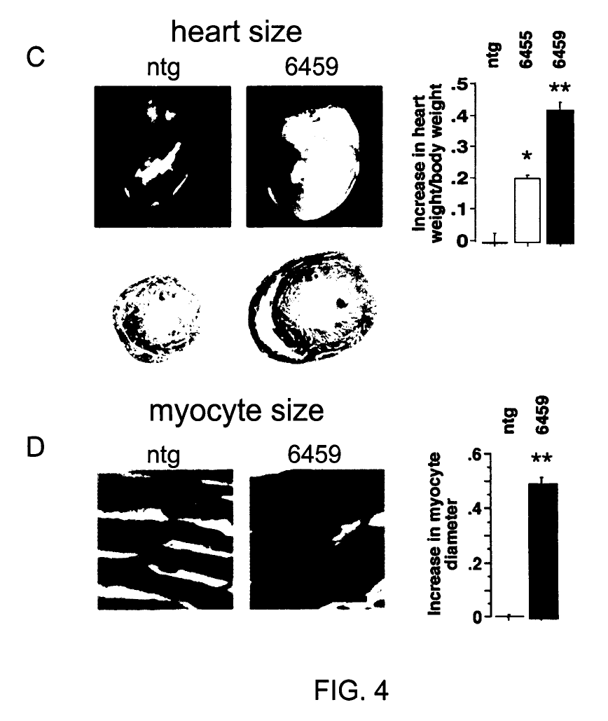

The normal down-regulation of Cdk9's activator, cyclin T1, was prevented in transgenic mice (FIG. 4) in order prove whether increased growth likewise would result in the intact heart from activation of endogenous Cdk9 at physiologically relevant levels. Transgene expression and its consequences are shown at the age of 2 months. Cyclin T1 levels and Cdk9 activity were increased ˜2 and 6-fold, in independent lines, similar to values in the early heart. The heart weight/body weight ratio increased 20% and 40%, respectively, with concentric hypertrophy. Myocyte diameter was assessed for the more highly expressing line, and was increased 50% compared to control littermates. Cardiac hypertrophy was induced by cyclin T1 without confounding apoptosis or fibrosis. Cyclin T protein and Cdk9 activity were both comparable to physiological levels in the younger heart (see FIG. 1A). By contrast, merely over-expressing Cdk9 had no effect on Cdk9 activation and no overt effect on cardiac growth.

### Example 8

### Cyclin T1 in Concert with Gq Provokes Rapidly Progressive Dilated Cardiomyopathy

Briefly, cardiac-specific (MHC-) cyclin T1 mice were crossed with the MHC-Gq line used to study endogenous Cdk9 activation by Gq. Cdk9 activity was increased synergistically by the combination of cyclin T1 (which activates Cdk9 directly) plus Gq (which dissociates Cdk9 from its inhibitor, 7SK snRNA). Mice inheriting both genes appear normal at birth and for the first week of life. However, by 3 weeks, the heart weight body weight ratio increases 73%, and growth retardation is obvious. By 4-5 weeks, bigenic mice begin to die, with progressive dilatation and thinning of the ventricular walls, and pleural effusions. Because direct biochemical data indicated that synergy of cyclin T1- and Gq-dependent pathways for Cdk9 activation suggested that Cdk9 may cause alternatively a benign or malignant cardiac phenotype, concentric versus dilated, depending on its level of activation. In each of these lines, Cdk9 kinase activity was mediated exclusively by the control of endogenous Cdk9, expressed at its own normal level. These phenotypes were reminiscent of the dosage-dependent effects of Gq.

### Example 9

### Cyclin T1 is Embryonic-Lethal Combined with Over-Expression of Cdk9

Briefly, four independent MHC-Cdk9 lines were made and high-level Cdk9 expression was confirmed by Western blotting. None increased Cdk9 activity, though, and none increased cardiac muscle growth. This suggested that activation of Cdk9, not the level of kinase was the limiting factor in cardiac muscle. Next, MHC-Cdk9 mice to were crossed with MHC-cyclin T1 mice. Typical Mendelian ratio of genotypes was shown at E10.5, but no bigenic animals at E12.5 or later. This strongly suggested that the T1×Gq cross resulted in excessive Cdk9 activity resulting in adverse consequences for the heart.

### Example 10

### Human Heart Samples

Human myocardium was obtained through The Methodist Hospital-DeBakey Heart Center. Heart failure tissue (Idiopathic dilated cardiomyopathy, DCM) was obtained from explanted hearts at the time of therapeutic transplantation. Normal hearts were obtained from unmatched organ donors and victims of motor vehicle accidents.

### Example 11

### CTD Kinase Expression and Function

Western blotting and immune complex kinase assays were performed as described (Sano et al. 2002). Association of P-TEFb with 7SK snRNA was determined by quantitative RT-PCR of 7SK snRNA from P-TEFb immune complexes, or Western-blotting of cyclin T1/Cdk9 using an RNA pull-down method, with biotinylated 2′-OMe oligonucleotide complementary to residues 221-241 of 7SK snRNA (Sano et al. 2002).

### Example 12

### Mouse Models

Cardiac-specific expression of dominant-negative human Cdk9 (D167N) was achieved using the mouse αMHC promoter (αMHC-dnCdk9), as detailed for αMHC-cyclin T1 (Sano et al. 2002). The conditional Mat1^{flox} mice were reported previously (Korsisaari et al. 2002). αMHC-Gq transgenic mice were provided by Dr. Gerald Dorn (D'Angelo et al. 1997). Pressure-overload cardiac hypertrophy was induced by transverse aortic banding (Oh et al. 2003); mice were used only in which constriction caused a right-to-left carotid flow velocity ratio of more than 4:1.

### Example 13

### Microarray Studies

Samples were labeled with biotinylated nucleotides by reverse transcription, hybridized to MG U74Av2 arrays (Affymetrix, San Jose, Calif.), and stained with streptavidin-phycoerythrin. Fluorescence intensities were captured with a ScanArray 5000 microarray scanner (Packard Bioscience, Meriden, Conn.), quantified with QuantArray software (GSI Lumonics, Watertown, Mass.), and analyzed using dChip 1.3 (Harvard University, Cambridge, Mass.).

### Example 14

### Quantitative RT-PCR

The same samples were subjected to quantitative RT-PCR using the ABI Prism 7700 sequence detection system (Perkin Elmer, Wellesley, Mass.) (Nakamura 2003). TaqMan primers and probes were designed using Primer Express software (version 1.0); details are available on request. For normalization, transcript levels were compared to glyceraldehyde-3-phosphate dehydrogenase.

### Example 15

### Histology

Hearts were fixed in 10% formalin overnight at 4° C., dehydrated with 70% ethanol, mounted in paraffin, and sectioned (5 μM). Sections were stained with hematoxylin and eosin or Gomori-trichrome. Myocyte diameter was measured using transnuclear width at the mid-ventricular level. For transmission electron microscopy, samples were prepared by standard procedures, sectioned using a RMC MT6000 ultramicrotome and visualized using a Hitachi H7500 electron microscope and 2K×2K Gatan CCD camera.

### Example 16

### Statistical Analyses

Data are reported as the mean±SE. Comparisons were analyzed by ANOVA, using a significance level of P<0.05.

### Example 17

### Pol II phosphorylation and Cdk9 Activity are Increased in Human Heart Failure

Because Cdk9 activity increased as both an acute and chronic response to hypertrophic signals in mouse myocardium, and sufficed to compel hypertrophic growth in transgenic mice (Sano et al. 2002), these data prompted us to speculate that prolonged increased in Cdk9 activity might contribute to the pathogenesis of heart failure. To examine whether our experimental models could pertain to human heart disease, ventricular myocardial samples from patients with heart failure due to dilated cardiomyopathy were examined (n=8) versus age-matched non-diseased hearts (n=8). Measured by an immune complex kinase assay with the recombinant CTD as substrate, Cdk9 activity increased 2.3±0.3 fold in human failing myocardium (p<0.05), and was increased in all eight hearts assayed. As in our studies of mice, Cdk9 activation occurred at unchanged levels of cyclin T1 and Cdk9 protein expression. Cdk7 activity likewise increased significantly (mean, 1.6±0.2 fold; p<0.05), but in only a minority of the samples.

### Example 18

### Cdk9 Activation Causes Late-Onset Heart Failure

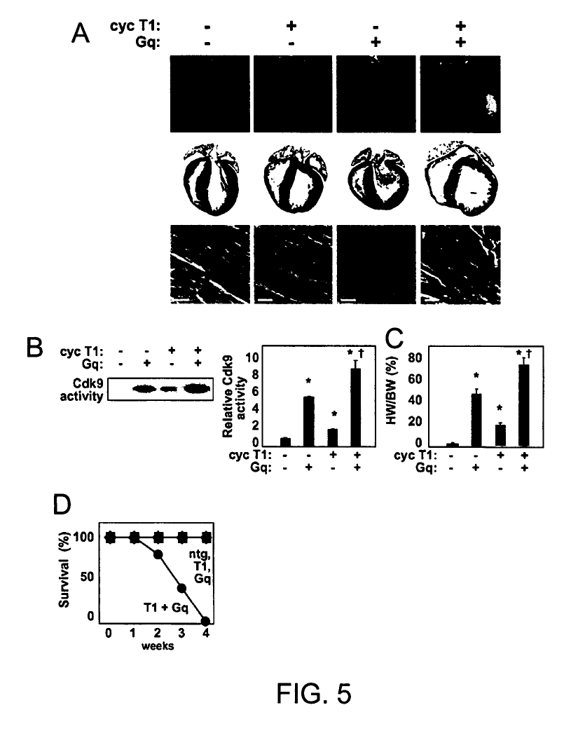

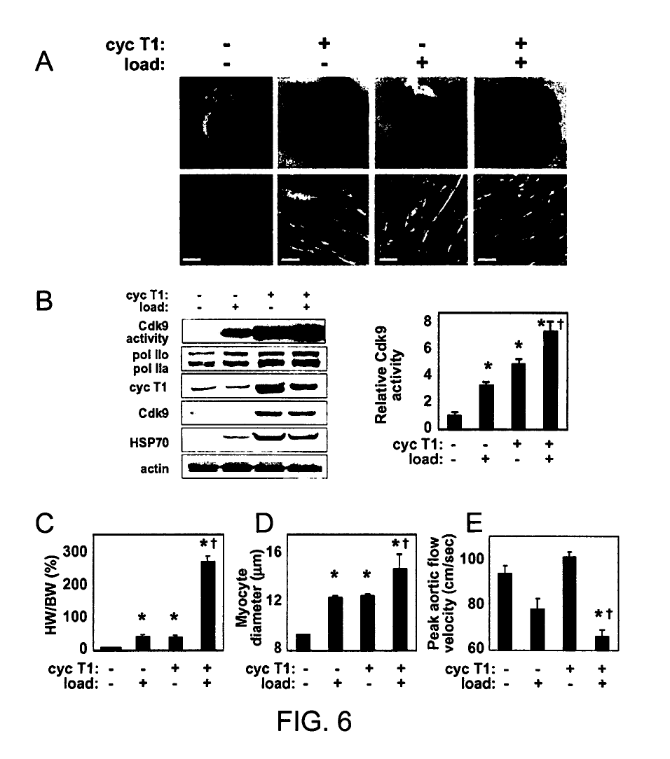

As an essential baseline for testing the impact of Cdk9 on cardiac adapation to complementary signals, a more comprehensive analysis was done of the αMHC-cyclin T1 mice (FIGS. 5, 6; Table 1). Immunoblotting confirmed that CTD phosphorylation was increased by transgenic expression of cyclin T1, as expected. By Doppler-echocardiography, cyclin T1 mice had normal left ventricular (LV) systolic function, compared with non-transgenic siblings, determined by fractional shortening and peak aortic flow velocity. Diastolic function at 3 months was well preserved, by comparison to αMHC-Gαq mice at the same age (Table 1) (D'Angelo et al. 1997).

TABLE 1
Doppler-echocardiographic assessment of mouse lines used for this study

| | non-transgenic | | |
|---|---|---|---|
| | littermates | αMHC-cyclin T1 | αMHC-Gq |
| N | 7 | 7 | 5 |
| HR (beats/min) | 409 ± 28 | 421 ± 34 | 292 ± 26* |
| LVEDD (mm) | 3.44 ± 0.12 | 3.98 ± 0.05* | 3.90 ± 0.08* |
| LVESD (mm) | 2.15 ± 0.12 | 2.3 ± 0.13 | 2.49 ± 0.10 |
| Fractional shortening | 38 ± 2% | 42 ± 3% | 36 ± 2% |
| Peak aortic velocity | 108 ± 3 | 109 ± 4 | 111 ± 7 |
| (cm · sec⁻¹) | | | |
| E/A ratio | 1.5 ± 0.1 | 2.6 ± 0.1*^{†} | 4.2 ± 0.4* |

*P < _vs ntg;
^{†}P < _vs αMHC-Gq

| | non-transgenic | αMHC- |
|---|---|---|
| | littermates | Cdk9 D167N |
| N | 7 | 14 |
| HR (beats/min) | 379 ± 18 | 345 ± 8 |
| LVEDD (mm) | 3.56 ± 0.18 | 3.33 ± 0.07 |
| LVESD (mm) | 2.28 ± 0.17 | 2.3 ± 0.07 |
| Fractional shortening | 36 ± 2% | 36 ± 2% |
| Peak aortic velocity | 108 ± 8 | 108 ± 3 |
| (cm · sec⁻¹) | | |
| E/A ratio | 1.4 ± 0.1 | 1.6 ± 0.1 |

*P < _vs ntg

| | αMHC-Cre^{+/o} MAT1^{L/+} | αMHC-Cre^{+/o} MAT1^{L/L} |
|---|---|---|
| N | 4 | 4 |
| HR (beats/min) | 346 ± 16 | 277 ± 40 |
| LVEDD (mm) | 4.01 ± 0.12 | 4.31 ± 0.33 |
| LVESD (mm) | 2.48 ± 0.2 | 3.79 ± 0.07* |
| Fractional | 39 ± 3 | 13 ± 4% |
| shortening | | |
| Peak aortic velocity | 101.5 ± 3.4 | 65.1 ± 8.9 |
| (cm · sec⁻¹) | | |
| E/A ratio | 1.7 ± 0.3 | 4.6 ± 0.4* |

*P < _vs control

High-throughput, unbiased measurements of gene expression were made with Affymetrix mouse oligonucleotide arrays and used to compare cyclin T1 mice and wild-type siblings at 3 months of age. Based on these results, QRT-PCR was then performed to confirm the microarray findings and survey additional pertinent genes. Notably, cyclin T1 mice did not express higher levels for many of the commonest markers of myocardial hypertrophy, such as brain natriuretic peptide (BNP), skeletal α-actin (SkA), and βMHC. By contrast, by either method, Hsp70 was up-regulated more than 10-fold, consistent with prior studies of pol II phosphorylation: specifically, “stalled” pol II is known to accumulate in the promoter-proximal region of the Hsp70 gene, and cyclin T/Cdk9 enables pol II to move into the Hsp70 open reading frame (Lis et al. 2000).

Conversely, forced expression of cyclin T1 down-regulated by 50% or more the genes for several pivotal cardiac proteins: αMHC, βMHC (ordinarily a marker of hypertrophy), the sarcoplasmic-endoplasmic reticulum calcium ATPase-2 (Atp2a2), cardiac ryanodine receptor (Ryr2), manganese superoxide dismutase (Sod2), and the gap junction protein connexin-43 (Cx43). Likewise, many nuclear-encoded mitochondrial genes related to energy synthesis (fatty acid metabolism, respiratory chain complexes) were down-regulated in cyclin T1 myocardium. Down-regulation of these two sets of genes was largely specific to cyclin T1 mice and not seen in the Gq model of hypertrophy at comparable age, despite even greater hypertrophy.

Among cardiac-specific transcription factors, Hand1 was markedly up-regulated, 39-fold compared with non-transgenic littermates. Conversely, the SRF co-activator myocardin (Wang et al. 2001) and homeodomain-only protein (HOP) (Chen et al. 2002) were downregulated in cyclin T1 mice, nearly 60 and 80% respectively. Unlike Hand2, Hand1 has not been shown consistently to serve as a transactivator, and in some reports serves instead as a transcriptional repressor (Bounpheng et al. 2000; Scott et al. 2000; Knofler et al. 2002). Hence, up-regulation of Hand1, down-regulation of these other cardiac factors, or both might contribute to the highly a typical program of gene expression in hypertrophy induced by cyclin T1. Notably, two transcription factors for mitochondrial biogenesis and function were also down-regulated in cyclin T1 mice: nuclear respiratory factor-1 and peroxisome proliferator-activated receptor-γ coactivator-1 (PGC-1), perhaps the best-proven candidate to explain coordinated down-regulation of mitochondrial enzymes in cardiac hypertrophy (Lehman et al. 2000; Czubryt et al. 2003).

Taken as an ensemble, these results indicate that latent biological dysfunction might exist in cyclin T1 transgenic mice, even though LV mechanical performance was largely sustained at 3 months of age. Consistent with this, LV dysfunction became overt in cyclin T1 mice beyond 1 year of age. Hence, hypertrophic growth induced through Cdk9 activity is ultimately more “pathological” than “physiological,” despite the absence of several common incriminating markers.

### Example 19

### Cdk9 Activation Predisposes Hearts to Decompensation Under Stress

As a provocative test for whether the net consequence of increased Cdk9 activity is adaptive or adverse, we crossed α-MHC-cyclin T1 transgenic mice to the α-MHC-Gq line (FIG. 5), or subjected them to pressure-overload by partial aortic constriction (FIG. 6). Each of these three states, independently, is a model of compensated concentric hypertrophy, and the mutual exacerbation by Gq plus mechanical stress is well known (Sakata et al. 1998).

Rapid ventricular dilatation and wall thinning resulted from the T1×Gq cross (FIG. 5A). Although cardiac Cdk9 activity is increased by Gq, further augmentation of Cdk9 activity resulted from coinheritance of the cyclin T1 transgene (FIG. 5B). The heart-to-body-weight ratio increased more than additively in double-transgenic mice (FIG. 5C), with heart failure and death ensuing invariably by just 4 weeks of age (FIG. 5D). On histological examination, the bigenic mice showed severe myofibril disarray and fibrosis throughout the myocardium (FIG. 5E).

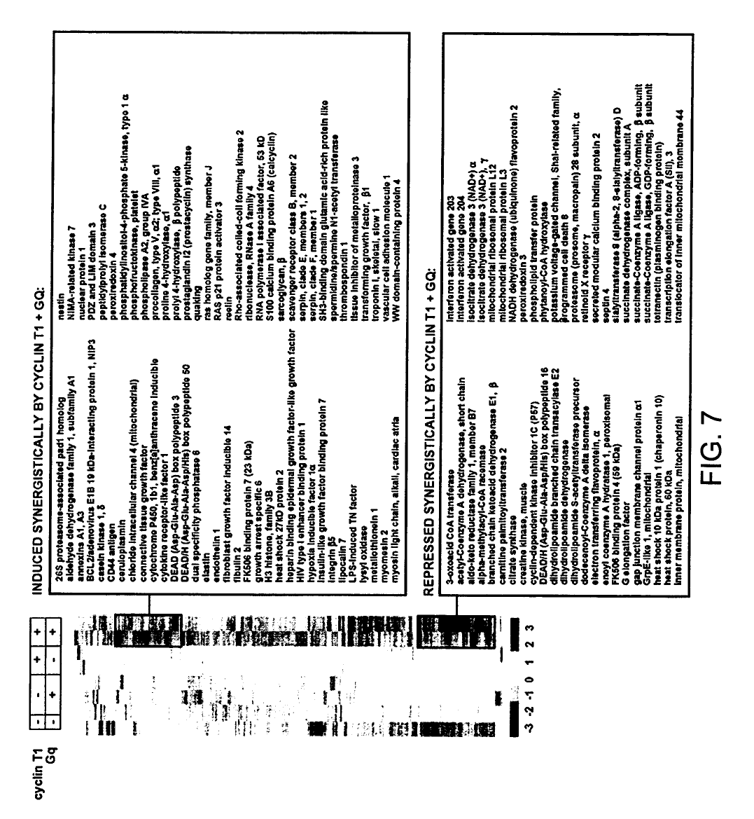

The molecular signature of heart failure was defined in this double transgenic model, and microarray and quantitative RT-PCR studies were performed (FIG. 7). Because of early lethality resulting from the genes' combined effect, ventricular RNA was compared at 2 weeks from non-transgenic, αMHC-cycT1, αMHC-Gq, and αMHC-cycT1/αMHC-Gq mice.

Expression of cyclin T1 and Gq in tandem may cause: (i) additive or synergistic effects on the same adverse genes, (ii) synergy by affecting distinct subsets of adverse genes, or (iii) a combination of these two mechanisms. By cluster analysis, examples of the first class were especially numerous. These include more than 60 genes induced only by the combination of cyclin T1 and Gq at the age examined, not by either gene alone. Among these were: (i) potential autocrine/paracrine factors (transforming growth factor β-1, heparin-binding epidermal growth factor, endothelin-1, connective tissue growth factor, growth arrest specific 6) (Asakura et al. 2002; Sano et al. 2002; Schultz et al. 2002; Candido et al. 2003; Nagai et al. 2003); (ii) intracellular signaling proteins of unknown function in hypertrophy (dual specificity phosphatase 6, NIMA-related kinase 7, phosphatidylinositol-4-phosphate 5-kinase 1αRho-associated coiled-coil forming kinase 2); mediators of apoptosis (Bnip3/Nix) (Yussman et al. 2002); (iii) transcription factors (hypoxia induced factor 1α, nuclear protein 1/p8); (iv) genes whose absence suffices for cardiomyopathy (PDZ and LIM domain 3, β-sarcoglycan) (Durbeej et al. 2000; Pashmforoush et al. 2001) and (v) markers of fibroblast activation (Col5a2, Col8a1, chloride intracellular channel 4). A much larger group showed roughly additive responses. Conversely, more than 40 genes were repressed as the consequence of cyclin T1 plus Gq yet not by either gene singly at this age, including many nuclear genes for mitochondrial biogenesis and fatty acid oxidation. Many of the microarray findings have already been confirmed by quantitative RT-PCR; PGC-1α was down-regulated 80% in the bigenic cyclin T1/Gq mice.

A corresponding analysis to test for functional interaction of cyclin T1 and mechanical stress was undertaken. As with cyclin T1 plus Gq, the effect on heart size was more than additive (FIG. 6A). Both genotypes received a comparable load, based on the right-to-left carotid artery flow velocity ratio after constricting the transverse aorta. Three weeks after banding, wild-type mice demonstrated a 3.0±0.2 fold increase in Cdk9 activity (p<0.05; FIG. 6B), with the heart-to-body-weight ratio increased 35±6% (p<0.05; FIG. 6C). Even though baseline Cdk9 activity was already 4.4±0.5 fold higher in cyclin T1 mice than in wild-type littermates (p<0.01), banding elicited a further 1.5±0.2 fold increase (p<0.01; FIG. 6B). In parallel with this combined effect on Cdk9, cyclin T1 transgenic mice showed even greater increase than non-transgenic littermates in the heart-to-body-weight ratio provoked by load (74.7±0.4%, p<0.001; FIG. 6C). Likewise, banding increased myocyte diameter from 9.3±0.1 μm to 12.6±0.1 μm in wild-type mice (p<0.001), and, roughly additively, from 12.7±0.1 μm to 15.1±1.2 μm in cyclin T1 mice (p<0.001; FIG. 6D).

Non-invasive echo-Doppler measurements are presently the means best suited to perform consecutive longitudinal studies of cardiac performance in mice, allowing each (before constriction) to serve as its own control (Oh et al. 2003). Cyclin T1 caused no decrement in baseline systolic function, but potentiated the load-induced fall. Peak aortic flow velocity decreased twice as much as in non-transgenic controls (before: 109.6±2.2 cm·sec⁻¹; after: 67.2±3.7 cm·sec⁻¹; p<0.05; FIG. 3E; see also Table 1). Post-operative lethality was prevalent in cyclin T1 mice (57.1%; n=7) but not non-transgenic littermates (0%; n=8).

Thus, although the baseline phenotype of αMHC-cyclin T1 mice is benign at 3 months of age, increased Cdk9 activity predisposes the myocardium to decompensation under stress imposed by two representative triggers for hypertrophy, pressure-overload and Gq.

### Example 20

### Catalytically Inactive Cdk9 Induces Spontaneous Heart Failure

If excess Cdk9 activation is adverse, a logical corollary is to predict that Cdk9 inhibition might be protective. In cell culture, Cdk9 was indispensable for pol II phosphorylation and myocyte growth after hypertrophic signals (Sano et al. 2002). However, the “ideal” level of activity in vivo is conjectural, perhaps no greater than the baseline in normal adult hearts, or perhaps needing to rise to some intermediary level (less than in cyclin T1 mice after stress, but more than in wild-type mice without stress).

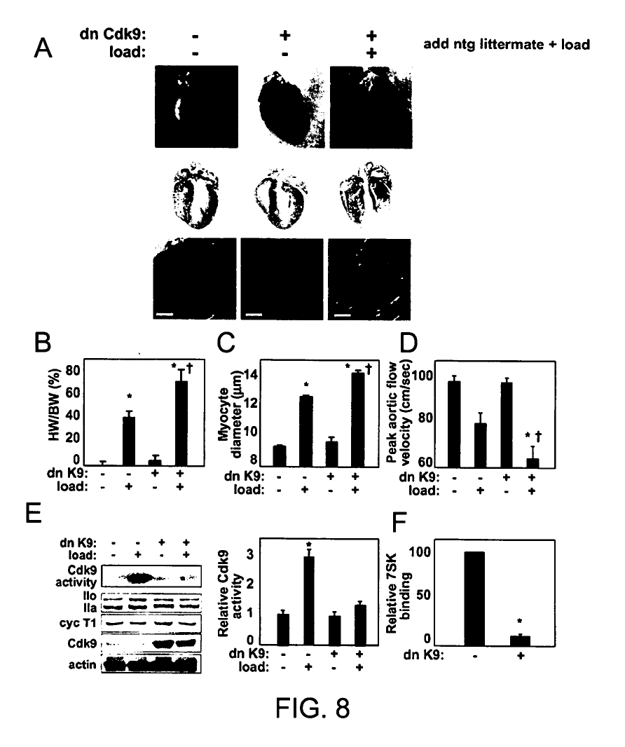

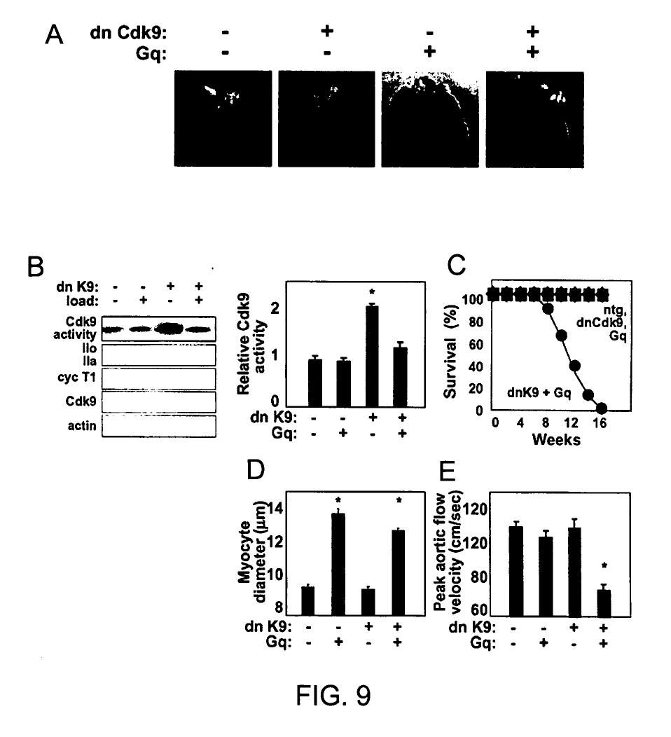

A generated cardiac-specific transgenic mice expressing catalytically inactive, dominant-negative Cdk9 (D167N) driven by the αMHC promoter (αMHC-dnCdk9; FIGS. 8, 9) was created in order to inhibit Cdk9 function. Three independent founders were obtained. First-generation (F1) heterozygous offspring of lines 8424 and 9433, the two highest expressers, all developed dilated cardiomyopathy with heart failure by 3-4 weeks of age and could not reproduce further. This exacerbation between the F0 and F1 phenotypes has been observed with other transgenes, ascribed to decreased tissue mosaicism in progeny of the founders (Zhang et al. 2000).

By contrast, the lowest expressing line showed no early lethality and was reproductively active (line 2542). For this line, the heart was morphologically normal at 3 months' age (FIG. 8A), with normal chamber size, wall thickness, and heart-to-body weight ratio (FIG. 8B), and no increase in fibrosis or apoptosis. Cardiomyocytes from dnCdk9 transgenic hearts had the same diameter as wild-type siblings' (FIG. 8C). Doppler-echocardiography revealed normal left ventricular dimensions and function (FIG. 8D).

Paradoxically, Cdk9 activity in ventricular myocardium was maintained at normal levels (FIG. 8E), although the level of dnCdk9 even in this lowest of the three transgenic lines was comparable to that after viral delivery in culture (Sano et al. 2002). Indeed, by co-precipitation, 7SK snRNA bound to the Cdk9-cyclin T1 complex was decreased by 90% in transgenic mice compared with wild-type ones (p<0.001; FIG. 8F).

Notwithstanding the lack of anatomical or cellular hypertrophy, dnCdk9 led to the activation of BNP, ANP βMHC, and skeletal α-actin to levels resembling the hypertrophic program in αMHC-Gq hearts.

### Example 21

### Catalytically Inactive Cdk9 Confers Intolerance to Stress

The hypothesis of whether kinase-deficient Cdk9 could prevent hypertrophy induced by Gq stimulation or chronic pressure overload was tested. αMHC-dnCdk9 mice (line 2542) were subjected to aortic banding (FIG. 8) or to mating with αMHC-Gq mice (FIG. 9).

Although baseline Cdk9 activity was preserved in dnCdk9 mice, the dnCdk9 transgene almost completely blocked the activation of Cdk9 provoked by load (FIG. 8E). Postoperative mortality increased markedly (55.6%; n=9), compared with banded non-transgenic littermates (0%; n=8). Surprisingly, the heart-to-body weight ratio increase after 21 days of load was nearly two-fold greater in dnCdk9 mice (57.8±9.0%; p<0.001) than in wild-type ones (FIG. 8B), with myocyte diameter increased equivalently (FIG. 8C), and impaired systolic function after banding (peak aortic flow velocity before constriction, 106.9±4.4 cm·sec⁻¹; after constriction, 65.0±7.3 cm·sec⁻¹; FIG. 8D; Table 1).

Analogously, co-inheriting dnCk9 and Gq evoked greater cardiac enlargement than Gq alone, with four-chamber enlargement and organized thrombus in the left atrium (FIG. 9A). Also, as with load, hypertrophic growth occurred even though transgenic expression of dnCdk9 blocked almost completely the signal-dependent increase in Cdk9 activity (FIG. 9B) and polII phosphorylation. Overt heart failure ensued at 8 weeks of age, with death invariably at 10-16 weeks of age, unlike the more benign phenotype from either transgene alone (FIG. 9B). At 6 weeks, myocyte drop-out and fibrotic replacement were observed in the atria, without obvious fibrosis or disarray in the ventricle. Left ventricular myocyte diameter increased 27% in double transgenic mice versus non-transgenic littermates, although less than in Gq mice (35%; FIG. 9D) Doppler-echocardiography demonstrated that peak aortic flow velocity was reduced only in double-transgenic mice (76.7±4.7 cm·sec⁻¹), not littermates of the other three genotypes (p<0.05; FIG. 9E). Thus, Gq and load caused hypertrophy in vivo despite both the presence of dnCdk9 and a successful block to the expected rise in Cdk9 activity. This transgenic phenotype differs notably from the effect of dnCdk9 in short-term culture and may be predicated, in part, on counter-regulatory effects of dissociating 7SK snRNA from the cyclin/Cdk complex (FIG. 9F); other, potential compensations would include mechanisms distal to transcript elongation, such as translational controls. Even more importantly, however, the level of block imposed markedly compromised the ability of the heart to adapt successfully to Gq signaling and mechanical stress. This, in turn, indicates that only less complete inhibition of Cdk9 could be beneficial (Sausville 2002).

### Example 22

### Cardiac-Specific Deletion of the Cdk7 Co-Factor MAT1 Causes Spontaneous Heart Failure

Conditionally-deleted essential cofactor MAT1 mice were engineered using Cre-lox technology (Gaussin et al. 2002; Korsisaari et al. 2002) to engineer the loss-of-function mutation in cardiac muscle. Germline deletion of MAT1 is embryonic-lethal before gastrulation (Rossi et al. 2001).

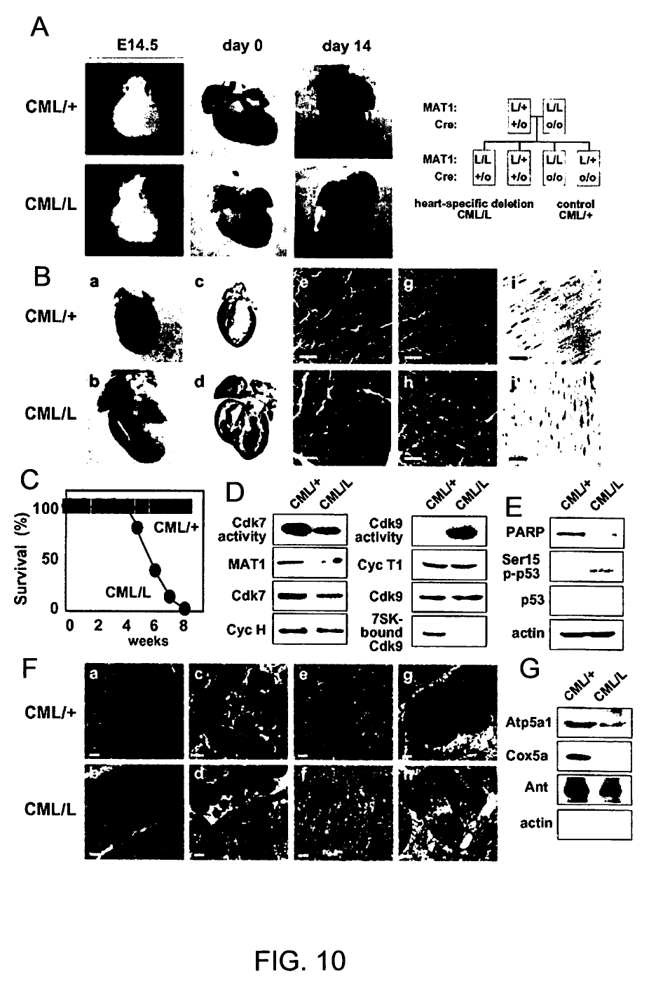

The αMHC-Cre^{+/-} MAT1^{lox/lox} mice (CML/L) were born with the expected Mendelian frequency, grew normally, and were undistinguishable from other littermates until 4 weeks of age (FIG. 10A). For comparisons, CML/+ mice are shown, bearing αMHC-Cre and one floxed MAT1 allele, but retaining one copy of the wild-type MAT1 gene. At 4 weeks, CML/L mice began to show decreased movement, dyspnea, or systemic edema. The hearts of CM^{L/L} mice were grossly enlarged with 4-chamber dilatation and atrial thrombi (FIG. 10B, panels a-d). Histologically, CML/L mice showed normal-sized muscle cells, mild fibrosis, and abundant TUNEL-positive myocytes (FIG. 10B, panels e-j). Echocardiography at 4 weeks revealed fractional shortening depressed by two-thirds (13±4%, versus 39±3% for the CML/+ control; p<0.001). Heart-specific deletion of MAT1 led inexorably to death by 6-8 weeks of age (FIG. 7C). This phenotype is reminiscent of the deletion of MAT1 selectively in Schwann cells, which was permissive for the normal mature phenotype but resulted ultimately in spontaneous cell degeneration (Korsisaari et al. 2002).

By both mRNA and protein expression (FIG. 10D), levels of MAT1 in ventricular myocardium of the CML/L mice decreased 60%, which is consistent with the expression of αMHC-Cre not just in cardiomyocytes but of MAT1 in all cells As seen with germline deletion of MAT1 (Rossi et al. 2001), Cdk7 and cyclin H protein expression also decreased (FIG. 10D), which may reflect the role of MAT1 as a chaperone for the cyclin H/Cdk7 complex. Cdk7 kinase activity decreased by 60% in CML/L mice compared with the CML/+littermate controls. Unexpectedly, Cdk9 kinase activity in 4 week-old CML/L mice increased more than 7-fold, independent of any change in Cdk9 or cyclin T1 expression (FIG. 10D). As with dnCdk9, this chronic counter-regulatory response involved release of the endogenous inhibitor, 7SK snRNA, from the cyclin T/Cdk9 P-TEFb complex.

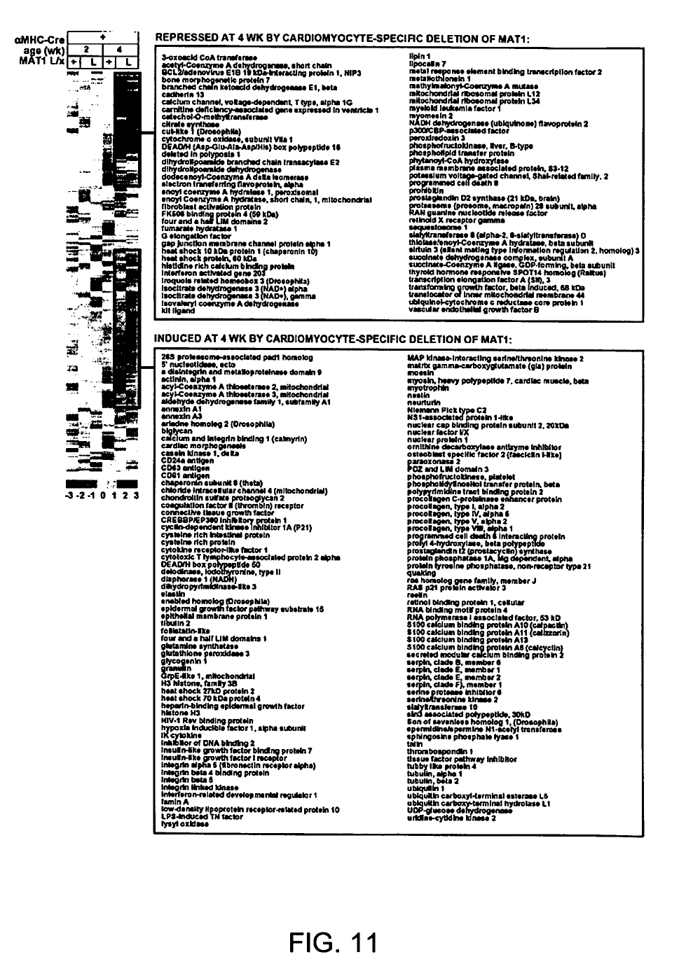

MAT1 disruption in a genetically unbiased way was studied in a genetically unbiased way and ventricular RNA from 2 and 4 week-old mice was subjected to microarray analysis, as done for the interaction of cyclin T1 and Gq (FIG. 11). At two weeks, no annotated genes were induced or repressed 50% or more by lack of MAT1, consistent with other evidence that MAT1 is dispensable for pol II-dependent transcription in mammalian cells (Rossi et al. 2001; Korsisaari et al. 2002; LeClerc et al. 2000). However, expression profiling in 4 week-old mice revealed the rapid evolution of changes in more than 400 genes, even at this stage where no lethality had occurred. Genes induced at 4 weeks by cardiomyocyte-specific disruption of MAT1 included (i) stress-associated proteins (Hsp27, Hsp70, hyoxia-inducible factor 1α), (ii) autocrine/paracrine factors and their binding proteins (heparin-binding epidermal growth factor, connective tissue growth factor, insulin-like growth factor I receptor), (iii) calcium-binding proteins (calcyclin, calizzarin, calpactin, calmyrin), (iv) focal adhesion and cytoskeletal proteins (alpha-actinin, alpha-actinin associated LIM protein, enabled, integrin β5, integrin linked kinase, talin, tubulin α1, tubulin β2), (v) other signal transducers (casein kinase 1δ, HIV-1 Rev binding protein, protein phophatase Mg-dependent 1a, protein tyrosine phosphatase non-receptor type 21, ras homolog gene family member J, RAS p21 protein activator 3, serine/threonine kinase 2, son of sevenless homolog 1), (vi) components of the the ubiquitin-proteasome pathway (adriadne, pad1, ubiquitin C-terminal esterase L5, ubiquitin C-terminal hydrolase L1), (vii) transcription factors (CREBBP/EP300 inhibitory protein 1, cysteine rich protein, four and a half LIM domains 1, inhibitor of DNA binding 2, RNA polymerase I associated factor 53, sin3-associated polypeptide 30), (viii) constituents and regulators of extracellular matrix (ADAM9, biglycan, elastin, fibulin, matrix Gla protein, multiple procollagen genes), and (ix) relatively few of the familiar hypertrophic markers (βMHC).

By contrast, the genes suppressed at 4 weeks by cardiomyocyte-specific disruption of MAT1 (FIG. 11) largely comprised genes for mitochondrial proteins—many for fatty acid oxidation and electron transport, but also the mitochondrial protein importer Tim44 (Rehling et al. 2001) and mitochondrial deacetylase Sirt3 (Onyango et al. 2002). Among regulators of transcription, expression decreased for transcription elongation factor A (TFIIS), which is necessary for “stalled” pol II to read through sites of transcription arrest (Pokholok et al. 2002). Also down-regulated was cut-like 1, which encodes an aytpical homeobox protein.

Based on the microarray findings, corroborative evidence of mitochondrial abnormalites was also found. By transmission electron microscopy (TEM), the myofibrils were poorly organized in 4 week-old mice with heart-specific deletion of MAT1, and mitochondria severely abnormal (more random distribution, irregular shape, decreased size, and fewer cristae; FIG. 10F, panels e-h). Even at 3 weeks, although myofibrils were properly aligned, abnormal mitochondria were already scattered among intact ones (FIG. 10F, panels a-d). Consistent with these structural observations, confirmation by immunoblotting showed down-regulation of ATP synthase α, the adenine nucleotide translocator (Ant), and cytochrome oxidase Va.

### Example 23

### Summary

CTD kinase activation and increased phosphorylation of pol II are hallmarks of human heart failure. This result reinforces the fidelity of the animal models to the human disease and the logic of Cdk9 and Cdk7, as a therapeutic target. Second, CTD kinase activation was proven directly to be adverse, causing florid heart failure when combined with other hypertrophic signals each of which is tolerated singly. This principle has been illustrated earlier by the grave effects of Gq combined with physiological instigators of heart growth (Sakata et al. 1998; Yussman et al. 2002). Cyclin T1 exacerbates both the load- and Gq-dependent phenotypes, leading to early lethality. Third, genetic inhibition of Cdk9 by a dominant-interfering mutation likewise was adverse. The harm that resulted from completely inhibiting the heart's ability to activate Cdk9 after stress is likely contingent on the extent of block imposed. Indeed, spontaneous lethality occurred in both lines with the inhibitor at even higher levels. Fourth, conditional deletion of the Cdk7 cofactor MAT1 unmasked an essential role for this protein in post-natal myocardium. The degenerative phenotype ensuing after birth resembles—but much sooner—the sequelae of deleting MAT1 in Schwann cells (Korsisaari et al. 2002).

Evidence has been herein provided for a striking rise in Cdk9 activity when MAT1 was deleted and Cdk7 activation impaired, as large as the inductive effect of load or of Gq. This occurred at 4 weeks, not two, and is presumed to be a secondary adaptation to deleting MAT 1, not a direct consequence.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Abdellatif, M. et al. Mol. Cell. Biol. 18, 6729-6736 (1998).

Adams, J. W. et al. Proc. Natl. Acad. Sci. U.S.A. 95, 10140-10145 (1998).

Agah, R. et al. J. Clin. Invest. 100, 169-179 (1997).

Akhtar, A., Faye, G. & Bentley, D. L. Embo J 15, 4654-4664. (1996).

Antos, C. L. et al. Proc. Natl. Acad. Sci. U.S.A. 99, 907-912 (2002).

Armache, K. J. et al. Proc Natl Acad Sci USA 100, 6964-6968 (2003).

Asakura, M. et al. Nat. Med. 8, 35-40. (2002).

Bounpheng et al. Exp Cell Res 257, 320-331 (2000).

Braasch, D. A. & Corey, D. R. Biochemistry 41, 4503-4510. (2002).

Bradsher, J et al. Mol Cell 10, 819-829 (2002).

Bueno, O. F. et al. EMBO J. 19, 6341-6350 (2000).

Candido, R et al. Circ Res 92, 785-792 (2003).

Chao, S. H., et al. J. Biol. Chem. 275, 28345-28348 (2000).

Chao, S. H. and D. H. Price. J Biol Chem 276, 31793-31799 (2001).

Chen, F., Cell 110, 713-723.

Cho, H. et al. Genes Dev. 13, 1540-1552 (1999).

Crackower, M. et al. Cell 110: 737 (2002).

Czubryt, M. P. et al. Proc Natl Acad Sci USA 100: 1711-1716 (2003).

Dahmus, M. E. J. Biol. Chem. 271, 19009-19012 (1996).

D'Angelo, D. D. et al. Proc. Natl. Acad. Sci. U.S.A. 94: 8121-8126 (1997).

de Falco, G. et al. Oncogene 19, 373-379 (2000).

De Luca, A. et al. J. Histochem. Cytochem. 49, 693-698 (2001).

Doi, T., et al. Biochemistry 38, 3090-3099 (1999).

Dorn, G. W. & Brown, J. H. Trends Cardiovasc. Med. 9, 26-34 (1999).

Durbeej, M. et al. Mol Cell 5: 141-151 (2000).

Egly, J. M. FEBS Lett 498: 124-128 (2001).

Esposito, G. et al. Circulation 105, 85-92 (2002).

Faust, M et al. Biochem Biophys Res Commun 296: 13-19 (2002).

Garry, D. J. et al. Trends Cardiovasc Med 13: 111-116 (2003).

Gaussin, V. et al. Proc. Natl. Acad. Sci. U.S.A. 99: 2878-2883 (2002).

Harper, J. W. and S. J. Elledge. Gene Develop 12: 285-289 (1998).

He, T. C. et al. Proc. Natl. Acad. Sci. U.S.A. 95, 2509-2514 (1998).

Herrmann, C. H. & Mancini, M. A. J Cell Sci 114, 1491-1503. (2001).

Hoque, M. et al. Mol. Cell. Biol. 23: 1688-1702 (2003).

Hunter, J. J. & Chien, K. R. N. Engl. J. Med. 341, 1276-1283 (1999).

Keriel, A. et al. Cell 109: 125-135 (2002).

Knofler, M. et al. Biochem J 361: 641-651 (2002).

Komamitsky, P. et al. Genes Dev 14: 2452-2460 (2000).

Korsisaari, N. et al. J Cell Sci 115: 4275-4284 (2002).

Kwak, Y. T., et al., J. Mol. Biol. 288, 57-69 (1999).

Lam, L. T. et al. Genome Biol. 2: research0041.0041-0041.0011 (2001).

Larochelle, S. et al. Embo J 20: 3749-3759 (2001).

Leclerc, V. et al. Embo J 19: 1567-1575 (2000).

Lehman, J. J. et al. J Clin Invest 106: 847-856 (2000).

Levy, D., et al. N. Engl. J. Med. 322, 1561-1566 (1990).

Lis, J. T., et al. Genes Dev 14: 792-803 (2000).

Litingtung, Y. et al. Mol Gen Genet 261: 100-105 (1999).

Lu, H. et al. Molecular & Cellular Biology 17: 5923-5934 (1997).

Lyons, G. E. et al. J. Cell. Biol. 111: 2427-2436 (1990).

MacLellan, W. R. & Schneider, M. D. Annu. Rev. Physiol. 62, 289-320 (2000).

Majello, B. & Napolitano, G. Front. Biosci. 6, D1358-1368 (2001).

Mathew, J. et al. Circulation 104, 1615-1621 (2001).

Matsuoka, M., et al., Mol. Cell. Biol. 14, 7265-7275 (1994).

McKinsey, T. A. & Olson, E. N. Curr. Opin. Genet. Develop. 9, 267-274 (1999).

Minamino, T. et al. Proc. Natl. Acad. Sci. U.S.A. 99, 3866-3871 (2002).

Molkentin, J. D. & Dorn, G. W., 2nd. Annu. Rev. Physiol. 63, 391-426 (2001).

Molkentin, J. D. et al. Cell 93, 215-228 (1998).

Mosterd, A. et al. N. Engl. J. Med. 340, 1221-1227. (1999).

Moteki, S. and D. H. Price. Mol Cell 10: 599-609 (2002).

Nagai, K. et al. J Biol Chem 278: 18229-18234 (2003).

Nakamura, T. et al. Proc. Natl. Acad. Sci U.S.A. 100: 5834-5839 (2003).

Newcomb, E. W. et al. Cell Cycle 2: 243-250 (2003).

Nguyen, V. T., et al., Nature 414, 322-325 (2001).

Oh, H. et al. Proc. Natl. Acad. Sci. U.S.A. 98, 10308-10313 (2001).

Olson, E. N. and M. D. Schneider. Genes Dev.: in press (2003).

Onyango, P. et al. Proc Natl Acad Sci USA 99: 13653-13658 (2002).

Orphamides, G. & Reinberg, D. Cell 108, 439-451. (2002).

Osuga, H. et al. Proc Natl Acad Sci USA 97: 10254-10259 (2002).

Pashmforoush, M. et al. Nat Med 7: 591-597 (2001).

Patturajan, M. et al. J Biol Chem 273, 4689-4694. (1998).

Peng, J., et al., Genes Dev. 12, 755-762 (1998).

Pokholok, D. K., N. M. Hannett, and R. A. Young. Mol Cell 9: 799-809 (2002).

Price, D. H. Mol Cell Biol 20: 2629-2634 (2000).

Rehling, P., N. Wiedemann, N. Pfanner, and K. N. Truscott. Mol Biol 36: 291-336 (2001).

Rossi, D. J., et al. Matl. Embo J 20: 2844-2856 (2001).

Sadoshima, J. & Izumo, S. Annu. Rev. Physiol. 59, 551-571 (1997).

Sakata, Y., B. D. Hoit, S. B. Liggett, R. A. Walsh, and G. Dorn. Circulation 97: 1488-1495 (1998).

Sandrock, B. and J. M. Egly. J Biol Chem 276: 35328-35333 (2001).

Sano, M. et al. Nat Med 8: 1310-1317(2002).

Sano, M. and M. D. Schneider. Cell Cycle 2: 99-104 (2003).

Sano, M. & Schneider, M. D. Circulation 105, 8-10 (2002).

Sausville, E. A. Mol Med 8: S32-37 (2002).

Schultz, J. E. et al. J. Clin. Invest. (2002)

Scott, I. C. et al. Mol Cell Biol 20: 530-541 (2000).

Shilatifard, A. et al. Annu. Rev. Biochem.: epub in advance of press (2003).

Shim, E. Y. et al. Genes Dev 16: 2135-2146 (2002).

Shioi, T. et al. EMBO J. 19, 2537-2548 (2000).

Shioi, T. et al. Mol. Cell. Biol. 22, 2799-2809. (2002).

Sinclair, A. M. et al. Blood 98: 3658-3667 (2001).

Subramaniam, A. et al. J. Biol. Chem. 266, 24613-24620 (1991).

Taigen, T., et al., Proc. Natl. Acad. Sci. U.S.A. 97, 1196-1201 (2000).

Verdecchia, P. et al. J. Am. Coll. Cardiol. 38, 1829-1835 (2001).

Wada, T. et al. Mol. Cell 5, 1067-1072 (2000).

Wallenfang, M. R. and G. Seydoux. Proc Natl Acad Sci USA 99: 5527-5532 (2002).

Wang, D. et al. Cell 105: 851-862 (2001).

Wettschureck, N. et al. Nat. Med. 7: 1236-1240 (2001).

Wimmer, J. et al. Virology 255, 182-189 (1999).

Yang, X., et al., J. Virol. 70, 4576-4584 (1996).

Yang, Z., et al., Nature 414, 317-322 (2001).

Yussman, M. G. et al. Nat. Med. 8: 725-730 (2002).

Zhang, D. et al. Nat. Med. 6, 556-563 (2000).

Zhou, M. et al. Mol. Cell. Biol. 20, 5077-5086 (2000).

Zhu, W. et al. J. Biol. Chem. 275, 15239-15245. (2000).

Zhu, Y. et al. Genes Dev. 11, 2622-2632 (1997).

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of treating a subject suffering from a cardiovascular disease comprising the step of administering to the subject an effective amount of a composition to modulate cyclin dependent kinase 9 (Cdk9) activity, wherein the effective amount modulates hypertrophic growth.

2. The method of claim 1, wherein the cardiovascular disease is heart failure.

3. The method of claim 1, wherein the composition comprises a Cdk9 inhibitor.

4. The method of claim 3, wherein the Cdk9 inhibitor is flavopiridol.

5. The method of claim 1, wherein the composition comprises a compound that modulates Cdk9 activity by prohibiting the dissociation of 7SK snRNA from cyclin T/Cdk9 complex.

6. The method of claim 5, wherein the composition comprises an inhibitor of Gq.

7. The method of claim 6, wherein the Gq inhibitor is selected from the group consisting of angiotensin II inhibitors, ACE inhibitors and endothelin inhibitors.

8. The method of claim 5, wherein the composition comprises an inhibitor of calcineurin.

9. The method of claim 8, wherein the calcineurin inhibitor is selected from the group consisting of angiotensin II inhibitors, ACE inhibitors and endothelin inhibitors.

10. The method of claim 1, wherein the composition comprises a compound that upregulates the levels of 7SK snRNA.

11. A method of modulating myocyte enlargement in a subject at risk for cardiac hypertrophy comprising the steps of administering to the subject an effective amount of a composition to modulate cyclin dependent kinase 9 (Cdk9) activity, wherein the effective amount modulates myocyte enlargement.

12. The method of claim 11, wherein the composition comprises a Cdk9 inhibitor.

13. The method of claim 12, wherein the Cdk9 inhibitor is flavopiridol.

14. The method of claim 11 wherein the composition comprises a compound that modulates Cdk9 activity by prohibiting the dissociation of 7SK snRNA from cyclin T1/Cdk9 complex.

15. A method of modulating cardiac hypertrophy comprising the step of administering to a subject an effective amount of a composition to modulate cyclin dependent kinase 9 (Cdk9) activity, wherein the effective amount modulates hypertrophic growth.

16. The method of claim 15, wherein the composition comprises a Cdk9 inhibitor.

17. The method of claim 16, wherein the Cdk9 inhibitor is flavopiridol.

18. The method of claim 15, wherein the composition comprises a compound that modulates Cdk9 activity by prohibiting the dissociation of 7SK snRNA from cyclin T/Cdk9 complex.

19. The method of claim 18, wherein the composition comprises an inhibitor of Gq.

20. The method of claim 19, wherein the Gq inhibitor is selected from the group consisting of angiotensin II inhibitors, ACE inhibitors and endothelin inhibitors.

21. The method of claim 18, wherein the composition comprises an inhibitor of calcineurin.

22. The method of claim 21, wherein the Gq inhibitor is selected from the group consisting of angiotensin II inhibitors, ACE inhibitors and endothelin inhibitors.

23. The method of claim 15, wherein the composition comprises a compound that upregulates the levels of 7SK snRNA.

24. A method of treating heart failure comprising the step of administering to a subject an effective amount of a composition to modulate cyclin dependent kinase 9 (Cdk9) activity.

25. The method of claim 24 further comprising administering calcium channel blocking agents, β-adrenergic blocking agents, angiotensin II inhibitors or ACE inhibitors.

26. A method of modulating a decrease in cardiac muscle contractile strength in a subject comprising the step of administering to the subject an effective amount of a composition to modulate cyclin dependent kinase 9 (Cdk9) activity, wherein the effective amount modulates the decrease in cardiac muscle contractile strength.

27. A method of treating a subject at risk for ventricular dysfunction associated with cardiac hypertrophy comprising the steps of administering to the subject an effective amount of a composition to modulate cyclin dependent kinase 9 (Cdk9) activity, wherein the effective amount decreases ventricular dysfunction.

28. A method of screening for a modulator of cyclin-dependent kinase 9 (Cdk9) comprising:
obtaining Cdk9;
contacting the Cdk9 with a candidate substance; and
assaying for Cdk9 activity, wherein when the Cdk9 activity changes after the contacting, the candidate substance is a modulator of Cdk9.

29. The method of claim 28, wherein the candidate substance inhibits Cdk9.

30. The method of claim 28, wherein the candidate substance prohibits the dissociation of 7SK snRNA from cyclin T/Cdk9 complex.

31. The method of claim 28, wherein assaying comprises RNA hybridization.

32. The method of claim 28, wherein assaying comprises PCR.

33. The method of claim 28, wherein assaying comprises RT-PCR.

34. The method of claim 28, wherein assaying comprises immunodetection.

35. The method of claim 34, wherein immunodetection comprises Western blot, ELISA or indirect immunofluorescence.


SEQUENCE LISTING (6 sequences)

SEQ ID NO: 1 - DNA, 21 bp, Artificial Sequence
  Synthetic Antisense
ccttgagagcttgtttggagg

SEQ ID NO: 2 - DNA, 20 bp, Artificial Sequence
  Synthetic Antisense
cgtttacgtcgccgtccagc

SEQ ID NO: 3 - DNA, 21 bp, Artificial Sequence
  Synthetic Primer
ggatgtgaggcgatctggctg

SEQ ID NO: 4 - DNA, 21 bp, Artificial Sequence
  Synthetic Primer
taaagaaaggcagactgccac

SEQ ID NO: 5 - RNA, 22 nt, Artificial Sequence
  Synthetic Antisense
accuugagagcuuguuuggagg

SEQ ID NO: 6 - DNA, 338 bp, Human
ggatgtgaggcgatctggctgcgacatctgtcaccccattgatcgccagggttgattcggctgatctggctggctaggcggtgtccccttcctccctcaccgctccatgtgcgtccctcccgaagctgcgcgctcggtcgaagaggacgaccttccccgaatagaggaggaccggtcttcggtcaagggtatagggtatacgagtagctgcgctcccctgctagaacctccaaacaagctctcaaggtccatttgtaggagaacgtagggtagtcaagcttccaagactgcagacacatccaaatgaggcgctgcatgtggcagtctgcctttcttta